(19) United States
(12) Patent Application Publication
(10) Pub. No.: US 2013/0064881 A1
(43) Pub. Date: Mar. 14, 2013

(54) COMPOSITIONS AND METHODS FOR TREATING PROSTATE CANCER

(71) Applicants: John J. Nemunaitis, Cedar Hill, TX (US); Donald Rao, Dallas, TX (US)

(72) Inventors: John J. Nemunaitis, Cedar Hill, TX (US); Donald Rao, Dallas, TX (US)

(73) Assignee: GRADALIS, INC., Carrollton, TX (US)

(21) Appl. No.: 13/607,546

(22) Filed: Sep. 7, 2012

Related U.S. Application Data
(60) Provisional application No. 61/532,403, filed on Sep. 8, 2011.

(51) Int. Cl. A61K 31/7088; A61K 9/127; A61K 39/395; G01N 33/566; A61K 31/711; A61P 35/00; A61N 5/10; C12N 15/63; C12Q 1/68; B82Y 5/00

(52) U.S. Cl. 424/450; 435/320.1; 514/44 A; 424/130.1; 435/6.12; 435/6.13; 600/1; 977/773; 977/906

(57) ABSTRACT

Compositions and methods to interfere with Androgen Receptor (AR) action based on bifunctional shRNA, targeting the AR and/or expression of SRC derived peptides are disclosed herein.

### CROSS-REFERENCE TO RELATED APPLICATIONS

This application claims priority to U.S. Provisional Application Ser. No. 61/532,403 filed on Sep. 8, 2011, the entire contents of which are incorporated herein by reference.

### TECHNICAL FIELD OF THE INVENTION

The present invention relates in general to the field of cancer therapy and more particularly, to compositions and methods for making and using bifunctional shRNA for a therapeutic RNA interference technology targeting Androgen Receptor (AR) as well as blocking the coactivator interface with AR by expressing coactivator derived peptide (SRCdp).

### STATEMENT OF FEDERALLY FUNDED RESEARCH

None.

### REFERENCE TO A SEQUENCE LISTING

The present application includes a Sequence Listing which has been submitted in ASCII format via EFS-Web and is hereby incorporated by reference in its entirety. Said ASCII copy, created on Sep. 4, 2012, is named GRAD1035_Sequence Listing.txt and is 19 KB in size.

### BACKGROUND OF THE INVENTION

Without limiting the scope of the invention, its background is described in connection with the gene therapies directed against prostate cancer and other maligancies.

U.S. Patent Application Publication No. 2010/0068802 (Qiu et al. 2010) relates to androgen receptor splice variants (AR3, AR4, AR4b, AR5 and AR8) and variants and fragments thereof which have a role in the progression of androgen independent prostate cancer. The invention further relates to compositions and methods that can be used to identify and treat prostate cancer.

U.S. Patent Application Publication No. 2005/0164970 (Li, 2005) discloses interfering RNA duplexes directed to the androgen receptor associated with prostate cancer. Also provided are methods of treating prostate cancer using interfering RNA duplexes to mediate gene silencing.

U.S. Patent Application Publication No. 2007/00873529 (Fletterick et al. 2007) relates to methods and antagonist compounds for modulating androgen receptor activity. Also provided is a method for identifying molecules that bind to a coactivator binding site of a receptor in the androgen receptor family. Also provided is a method for inhibiting androgen receptor activity in a mammal, thereby facilitating treatment of diseases such as prostate cancer.

### SUMMARY OF THE INVENTION

The present invention includes compositions and methods for making and using bifunctional shRNA for a therapeutic RNA interference technology targeting Androgen Receptor (AR) as well as p160 coactivator-derived peptides (SRCdp) that block the coactivator interface.

In one embodiment the instant invention discloses a vector comprising: a first promoter; and a nucleic acid insert operably linked to the promoter, wherein the insert encodes one or more short hairpin RNAs (shRNA) capable of inhibiting an expression of an AR gene. In one aspect the shRNA is a bifunctional shRNA, wherein the shRNA comprises one or more siRNA (cleavage-dependent) and miRNA (cleavage-independent) motifs. In another aspect the shRNA is both a cleavage-dependent and cleavage-independent inhibitor of the AR gene. In another aspect a sequence arrangement for the shRNA comprises a 5′ stem arm-19 nucleotide target (AR gene)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′stem arm.

In yet another aspect the one or more shRNA correspond to a human and a mouse AR gene, wherein the one or more shRNA are capable of inhibiting an expression of a human and a mouse AR gene. In a related aspect the one or more shRNAs are selected from the group consisting of SEQ ID NO: 10, SEQ ID NO: 11, SEQ ID NO: 12, SEQ ID NO: 13, and any combinations or modifications thereof. In another aspect further comprising a second nucleic acid insert operably linked to a second promoter, wherein the second insert encodes SRCdp, wherein the SRCdp is capable of blocking the AR-coactivator interface. In yet another aspect the first promoter and the second promoter are the same promoter and wherein an optimum gap sequence is intercalated between the first and the second nucleic acid inserts.

Another embodiment disclosed herein relates to an expression vector comprising: a promoter; and a nucleic acid insert operably linked to the promoter, wherein the insert encodes a coactivator-derived peptide (SRCdp), wherein the SRCdp is capable of blocking a AR-coactivator interface. In a specific aspect SRCdp is derived from SRC-1. In another aspect SRCdp is derived from human or mouse SRC-1 and is capable of blocking a human and a mouse AR-coactivator interface. In another aspect the SRCdp comprises amino acids 1050-1240 of SRC-1 (SEQ ID NO: 16) or amino acids 1050-1150 (SEQ ID NO: 14) of SRC-1. In a specific aspect SRCdp is selected from SEQ ID NO. 15, SEQ ID NO: 15, or both. In another aspect SRCdp is derived from SRC-1, SRC-2, or SRC-3 and further comprises a nuclear localization signal fused to SRCdp. In yet another aspect the present invention comprises a second nucleic acid insert operably linked to a promoter, wherein the second insert encodes one or more short hairpin RNAs (shRNA) capable inhibiting an expression of a AR gene.

In yet another embodiment the instant invention discloses a therapeutic delivery system comprising: a therapeutic agent carrier; and a vector that binds prostate cells comprising a first nucleic acid insert operably linked to a first promoter or a second nucleic acid insert operably linked to a second promoter or combinations thereof, wherein the first nucleic acid insert encodes one or more short hairpin RNAs (shRNA) capable of inhibiting an expression of a AR gene, wherein the second nucleic acid insert encodes a SRCdp capable of blocking a AR-coactivator interface. In one aspect the first promoter and the second promoter is the same promoter and wherein an optimum gap sequence is intercalated between the first and the second nucleic acid inserts. In another aspect the therapeutic agent carrier is a nanoparticle capable of compacting DNA, wherein the nanoparticles comprise one or more polycations. In another aspect the delivery system further comprises one or more 10 kDA polyethylene glycol (PEG)-substituted cysteine-lysine 3-mer (CK30PEG10k) peptides. In yet another aspect the compacted DNA nanoparticles are further encapsulated in a liposome, wherein the liposome is a bilamellar invaginated vesicle (BIV).

In one aspect the liposome is a reversibly masked liposome. In another aspect the liposome is decorated with one or more “smart” receptor targeting moieties, wherein the one or more “smart” receptor targeting moieties are small molecule bivalent beta-turn mimics. The skilled artisan would understand that these targeting moieties can include other types of proteins, (for e.g. glycoproteins, and other structurally and functionally related proteins) and may also include non-protein receptors, etc. In yet another aspect the delivery system is adapted for use to suppress tumor cell growth, treat prostate cancer, or both in a human or animal subject by itself or in combination with one or more chemotherapeutic agents, radiation therapy, surgical intervention, antibody therapy, Vitamin D, or any combinations thereof.

In one aspect the instant invention provides a method to deliver a vector to a tissue comprising: i) providing a vector comprising a first nucleic acid insert operably linked to a first promoter, or a second nucleic acid insert operably linked to a second promoter, or combinations thereof, wherein the first nucleic acid insert encodes one or more short hairpin RNAs (shRNA) capable of inhibiting an expression of an AR gene and wherein the second nucleic acid insert encodes a SRCdp capable of blocking a AR-coactivator interface, ii) combining the expression vector with a therapeutic agent carrier, and iii) administering a therapeutically effective amount of the expression vector and therapeutic agent carrier complex to a patient in need thereof.

A method of suppressing a tumor cell growth, treating prostate cancer, or both in a human subject is also disclosed in one embodiment of the present invention. The method comprises the steps of: identifying the human subject in need for suppression of the tumor cell growth, treatment of prostate cancer or both and administering a vector in a therapeutic agent carrier complex to the human subject in an amount sufficient to suppress the tumor cell growth, treat prostate cancer or both, wherein the vector comprises a first nucleic acid insert operably linked to a first promoter, or a second nucleic acid insert operably linked to a second promoter, or combinations thereof, wherein the first nucleic acid insert encodes one or more short hairpin RNAs (shRNA) capable of inhibiting an expression of a AR gene and wherein the second nucleic acid insert encodes a SRCdp, wherein the SRCdp is capable of blocking a AR-coactivator interface, wherein inhibition of AR expression or blockage of the AR-coactivator interface reduces tumor growth.

In one aspect the method as described hereinabove further comprises the step of administering the vector before, after, or concurrently as a combination therapy with one or more treatment methods selected from the group consisting of chemotherapy, radiation therapy, surgical intervention, antibody therapy, Vitamin D therapy, or any combinations thereof. In another aspect the therapeutic agent carrier is a nanoparticle capable of compacting DNA or a reversibly masked liposome decorated with one or more “smart” receptor targeting moieties.

Another embodiment disclosed herein relates to a method for studying biological and clinical manifestations of a current or proposed anti-cancer therapeutic strategy in a human or animal subject, customizing anti-cancer therapy for an individual human or animal subject, or both in a human or animal subject comprising the step of: i) identifying the human or animal subject in need of screening for reactions to an anti-cancer medication, customization of the anti-cancer therapy, or both, ii) administering a vector in a therapeutic agent carrier complex to the human or animal subject in an amount sufficient to suppress the tumor cell growth, cancer or both, wherein the vector comprises a first nucleic acid insert operably linked to a first promoter, or a second nucleic acid insert operably linked to a second promoter, or combinations thereof, wherein the first nucleic acid insert encodes one or more short hairpin RNAs (shRNA) capable of inhibiting an expression of a AR gene and wherein the second nucleic acid insert encodes a SRCdp, wherein the SRCdp is capable of blocking a AR-coactivator interface, wherein inhibition of AR expression or blockage of the AR-coactivator interface reduces tumor growth, iii) collecting biological and clinical information from the human or animal subject after administration of the vector in the therapeutic agent complex, and iv) making a decision to terminate, continue, or modify a current or proposed anti-cancer therapeutic strategy in the human or animal subject based on the biological or clinical information, wherein the therapeutic strategy comprises administration of the vector in the therapeutic agent carrier by itself or in combination chemotherapy, radiation therapy, surgical intervention, antibody therapy, Vitamin D therapy, or any combinations thereof. In a specific aspect of the method herein the cancer is prostate cancer.

### BRIEF DESCRIPTION OF THE DRAWINGS

For a more complete understanding of the features and advantages of the present invention, reference is now made to the detailed description of the invention along with the accompanying figures.

FIG. 1 depicts the structure of SRC-1. The location of the AR interacting peptides P200 and P100 in the Q rich domain are depicted as are the LXXLL motifs, which are required for binding of other steroid receptors, but not for AR.

FIG. 2 demonstrates that P100 and P200 inhibit AR activity with little effect on GR. HeLa cells were transfected with an AR/GR responsive reporter, AR or GR expression plasmids and increasing amounts of P100 or P200 and hormone dependent activity measure. P100 has no effect on GR activity and the amount of P200 required to block AR activity has no effect on GR although higher levels are slightly inhibitory.

FIGS. 3A-3E documents that SRCdp inhibits AR but not VDR activity. Cells were transfected with vector or SRCdp treated with androgen (R1881) (panel A, B, C) or 1,25-dihydroxyvitamine D3 (1,25D) in panel E 24 hrs post transfection and harvested 24 hrs later. Panel D, C4-2 cells were transfected with vector or SRCdp. RNA was extracted and analyzed by quantitative RTPCR. The horizontal line represents the expected level of activity if 100% inhibition were achieved in the 50% of cells transfected.

FIG. 4 shows that P200 blocks expression of PSA. LNCaP cells were transiently transfected with vector or P200 plasmid, nuclei detected with DAPI, PSA with FITC anti-PSA and P200 with anti-flag and Texas red. Note that the Texas Red positive cells contain almost no PSA.

FIG. 5 documents the effect of SRCdp on cell proliferation. Cells were transfected with pCR3.1, or SRCdp(P100). Proliferation was measured by [3H] thymidine incorporation 48 hrs later.

FIGS. 6A-6C shows inducible expression of the AR splice variant (AR-V7) in a stably transfected LNCaP cell line. All studies were carried out in media containing charcoal-stripped serum. Quantitative results were analyzed using one-way ANOVA and LSD post-hoc comparison (*, p<0.05): FIG. 6A shows Western blot analysis of AR isoform expression in cells treated with doxycycline (Dox) or R1881), FIG. 6B illustrates studies in which cells were treated in parallel with those in Figure A, and PSA mRNA expression was quantified by qPCR, and FIG. 6C illustrates studies in which cell growth was measures at 72 and 110 hours following treatment with Dox, alone or in combination with R1881.

FIGS. 7A and 7B are schematic representations showing the design of the bi-functional shRNAs of the present invention. FIG. 7A shows the sequence arrangement for a single target and FIG. 7B shows the sequence arrangement for multiple targets.

FIGS. 8A-8D show plasmid circular maps for different candidate bi-sh-AR constructs: FIG. 8A bi-shRNA-hAR1 (pGBI-100), FIG. 8B bi-shRNA-hAR2 (pGBI-101), FIG. 8C bi-shRNA-hAR3 (pGBI-102), and FIG. 8D bi-shRNA-hAR4 (pGBI-103).

FIGS. 9A and 9B are plasmid maps of two fragments of SRC-1, one containing approximately 200 amino acids (FIG. 9B) and one containing 100 amino acids (FIG. 9A).

FIGS. 10A and 10B are plasmid maps corresponding to the 100 amino acid fragment (pNLSp100) and the 200 amino acid fragment (pNLSp200), respectively.

FIG. 11 depicts regions corresponding to P100 and P200 on SRC-1. The LXXLL motifs are required for interaction of SRC-1 with other steroid receptors. The glutamine (Q) rich region, amino acids 989-1240 contains the region which interacts with AR.

FIG. 12 depicts predicted Stem-loop structure of bi-shRNA Targeting AR. The bifunctional shRNA (bi-shRNA) consists of two stem-loop structures to facilitate loading onto multiple RISCs leading to both RNA degradation, sequestration and translational repression.

FIGS. 13A and 13B show that P100 and P200 interact with androgen receptor and inhibit the AR and SRC-1 interaction. FIG. 13A depicts mammalian two hybrid assay demonstrating that SRC-1 and the two peptides linked to a Gal binding domain interact with full length AR. Interaction is measuring using 17-mer luc Gal responsive reporter. FIG. 13B shows results of experiments to compare the relative efficacy of full length SRC-1 and P100 and P200 in reducing the interaction between SRC-1 and AR. A mammalian two hybrid assay as in FIG. 13A was performed except that increasing amounts of plasmid encoding SRC-1, P100, or P200 were added during the transfection. In all cases, plasmid was balanced with parent vector. Note that P100 and P200 are as effective at blocking the interaction as is the full length SRC-1.

FIG. 14 shows that peptides inhibit activity of AR as well as of the AR-V7 splice variants. HeLa cells were transiently transfected with plasmids encoding AR (left) or AR-V7 (right), an AR responsive luciferase reporter, and increasing levels of P100 or P200 plasmid balanced with vector DNA. AR cells were treated with vehicle or R1881 and cells harvested the next day. Both P100 and P200 inhibit AR and AR-V7 activity.

FIG. 15 shows target and receptor-specific effects of peptides. LNCaP cells were transfected with vector, P100 or P200 plasmid, treated with vehicle, R1881 or 1,25-dihydroxyvitamin D3 (1,25D) for 24 hours, RNA isolated, target genes measured by real time RT-PCR and normalized to 18S. The transfection efficiency was approximately 50% and the red line indicates the predicted remaining level of expression if the AR in the transfected cells were completely inhibited. Note that the induction of the two AR target genes is inhibited, but the peptides do not prevent AR dependent repression or vitamin D receptor dependent induction of CYP24A1 suggesting that the peptides show specificity for AR.

FIG. 16 shows that peptides inhibit proliferation of AR dependent LNCaP and C4-2 cells, but not AR negative PC-3 cells. LNCaP and PC-3 cells were transfected with vector (pCR3.1), P100, or P200, re-plated after 48 hours, treated with vehicle or R1881 overnight and proliferation measured by [3H] thymidine incorporation. C4-2 cells at 20% confluency were transfected with vector, P100 or P200. After 24 hours, cells were treated with vehicle or hormone for another 48 hours. Proliferation was measured by [3H] thymidine incorporation.

FIGS. 17A and 17B show bi-shAR mediated AR depletion in HeLa cells. HeLa Cells were transfected with an AR expression plasmid and various amounts of vector or AR targeted bi-shRNA. In FIG. 17A cells were harvested after 24 or 48 hours, cell lysates prepared and AR expression determined by western blotting. FIG. 17B: a 72 hour time course with the best shRNA.

FIG. 18A, 18B, 18C, 18D show Bi-shAR103 mediated depletion of endogenous AR in LNCaP cells. Bi-shAR103Mediated Depletion of Endogenous AR in LNCaP Cells. Cells were electroporated with vector or bi-shAR103, harvested at the indicated times, and AR detected by western blotting (FIG. 18A) or RNA measured by real time RT-PCR (FIG. 18B) or RNAi mediated cleavage product detected by RNA Ligase-Mediated RT-PCR (FIG. 18C). LNCaP cells plated on lysine-coated coverslips were co-transfected with bi-shAR103 or vector and a GFP expression plasmid at a 50:50 ratio using XtremeGene HP reagent. After 48 hours cells were fixed with formaldehyde, incubated with an AR antibody, and AR detected using Alexafluor 594 conjugated secondary antibody and a DeltaVision Image Restoration Microscope. Note in the first row that while AR is readily visible in GFP and vector co-transfected cells, cells in the second row co-transfected with GFP and bi-shAR103 show markedly less AR fluorescence than the surrounding cells (FIG. 18D).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Androgens bind to and activate the androgen receptor (AR), a hormone activated transcription factor, and treatments that reduce circulating androgens are the primary treatment for metastatic prostate cancer. Tumors that respond initially typically become refractory to treatment within two years. However, the present inventors recognize that most castration resistant tumors remain AR dependent. Among the potential changes that contribute to the elimination of the requirement for normal circulating levels of androgens are increased AR expression and altered cell signaling sensitizing AR to lower levels of hormone. Additionally, local synthesis of androgens can contribute to AR activation. The recent discovery of constitutively active AR splice variants that lack the AR hormone binding domain highlights the need for new treatments that either reduce AR expression or target other functional domains of AR. Unlike most steroid receptors, the primary coactivator binding site for AR resides in the amino-terminus, which is retained in the splice variants. The present inventors recognize that that occluding the coactivator binding site would be sufficient to block AR activity and have identified a region of the p160 coactivator, SRC-1, which is sufficient to block induction of target genes by AR as well as AR dependent cell growth of both hormone dependent and castration resistant prostate cancer cells. This region has no effect on growth of AR negative PC-3 prostate cancer cells and little effect on the activity of other nuclear receptors including the glucocorticoid and thyroid receptors. Thus, expression of this region has potential therapeutic utility in prostate cancer. A second approach is to reduce expression of AR. The present inventors recognize that major advantage of both of these approaches is that eliminating AR or its activity has minimal consequences in other tissues. To reduce AR expression the present inventors designed four candidate bi-shRNAs. The present inventors recognize that bifunctional shRNA technology demonstrates a more effective silencing of target gene expression by concurrently inducing translational repression, mRNA sequestration in the p body as well as post-transcriptional mRNA cleavage. The present inventors tested the efficacy and potency of the four shRNAs by co-expressing AR and the shRNAs in HeLa cells and found that the potency and efficacy of the four differed substantially. The present inventors have tested the best in the LNCaP prostate cancer cell line. The net reduction in AR expression is consistent with the relative transfection efficiency of these cells. Co-transfection of a GFP expression vector and the bi-shRNA followed by detection of AR by secondary immunofluorescence suggests that the successfully transfected cells lose essentially all AR expression. The present inventors also optimize the SRC-1 fragment. The optimal bi-shRNA and SRC-1 fragments are tested for efficacy in xenograft models using tail vein delivery of lipoplexes (expression plasmid complexes involving bilamellar invaginated liposomes).

The present inventors also found that SRC derived peptides, P100 and P200, specifically inhibit AR dependent gene induction and AR dependent cell growth, tested four bi-shRNAs targeting AR, wherein bi-sh103 was significantly more effective than the other three bi-sh103 reduced AR expression as measured by western blotting, qRT-PCR and RLM-RT PCR. The apparent partial reduction in LNCaP cells was a result of transfection efficiency, and cells co-transfected with bi-sh103 and a GFP plasmid were AR negative when assayed by immunofluorescence whereas vector control/GFP cells retained AR expression.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “expression vector” as used herein in the specification and the claims includes nucleic acid molecules encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter. The term “promoter” refers to any DNA sequence which, when associated with a structural gene in a host yeast cell, increases, for that structural gene, one or more of 1) transcription, 2) translation or 3) mRNA stability, compared to transcription, translation or mRNA stability (longer half-life of mRNA) in the absence of the promoter sequence, under appropriate growth conditions.

The term “oncogene” as used herein refers to genes that permit the formation and survival of malignant neoplastic cells (Bradshaw, T. K.: Mutagenesis 1, 91-97 (1986).

As used herein the term “receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

The term “hybridizing” refers to any process by which a strand of nucleic acid binds with a complementary strand through base pairing.

The term “transfection” refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

As used herein the term “bi-functional” refers to a shRNA having two mechanistic pathways of action, that of the siRNA (cleavage-dependent RISC loading) and that of an miRNA-like moiety (cleavage-independent RISC loading and target mRNA complementarity). A bifunctional construct concurrently represses the translation of the target mRNA, facilitates mRNA degradation and p-body mRNA sequestration, and cleaves target mRNA through RNase H-like cleavage.

The term “traditional” shRNA refers to a DNA transcription derived RNA acting by the siRNA mechanism of action. The term “doublet” shRNA refers to two shRNAs, each acting against the expression of two different genes but in the “traditional” siRNA mode.

As used herein, the term “liposome” refers to a closed structure composed of lipid bilayers surrounding an internal aqueous space. The term “polycation” as used herein denotes a material having multiple cationic moieties, such as quaternary ammonium radicals, in the same molecule and includes the free bases as well as the pharmaceutically-acceptable salts thereof.

The term “pharmaceutically acceptable” indicates that the carrier, diluent or excipient must be compatible with the other ingredients of the formulation and not deleterious to the recipient thereof.

The terms “administration of” or “administering a” compound is understood in the art to indicate providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” as used herein refers to the amount of the subject compound that will elicit the biological or medical response of a tissue, system, animal or human that is being sought by the researcher, veterinarian, medical doctor or other clinician.

As used herein, the term “treatment” or “treating” indicates any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The vector of the present invention in a therapeutic agent carrier may be administered simultaneously or sequentially in one or a combination of dosage forms. by subcutaneous, intravenous, intraperitoneal, etc., administration (e.g. by injection). It can be administered with one or more chemotherapeutic agents, with radiation, surgical treatment, antibody therapy, or any combinations thereof.

To administer the therapeutic compound of the present invention by any other route other than parenteral administration, it may be necessary to coat the compound with, or co-administer the compound with, a material to prevent its inactivation. For example, the therapeutic compound may be administered to a subject in an appropriate carrier, for example, emulsifiers, liposomes, or a diluent. Pharmaceutically acceptable diluents include saline and aqueous buffer solutions. In all cases, the composition must be sterile and must be fluid to the extent that easy syringability exists. It must be stable under the conditions of manufacture and storage and must be preserved against the contaminating action of microorganisms such as bacteria and fungi.

Sterile injectable solutions can be prepared by incorporating the therapeutic compound of the present invention in the required amount in an appropriate solvent with one or a combination of ingredients enumerated above, as required, followed by filtered sterilization.

Techniques and compositions for making useful dosage forms using the present invention are described in one or more of the following references: Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), and the like, relevant portions of each incorporated herein by reference.

A parenteral composition suitable for administration by injection is typically prepared by stirring sufficient active ingredient in deionized water and mixed with, e.g., up to 10% by volume propylene glycol, salts and/or water to deliver a composition, whether in concentrated or ready-to-use form. The solution will generally be made isotonic with sodium chloride and sterilized using, e.g., ultrafiltration.

The present invention includes compositions and methods related to inhibiting AR action and therapy for prostate tumor growth regardless of the mode of AR activation.

Due to the minimal toxicity, the present disclosure is useful for the treatment of castration resistant prostate cancers (CRPC), and tumors that are still responsive to androgen blockade. Some embodiments of the invention are delivered in combination with conventional androgen blockade. In further embodiments, it is used as a single treatment. The present inventors recognize that the present disclosure provides a way to prevent bone and muscle loss, sexual side effects of overall androgen ablation, and major benefits for the quality of life of the patients because AR action can be blocked without unacceptable side effects in prostate cancer patients.

The present inventors recognize that primary prostate cancer is an androgen-dependent disease and that the actions of androgens, primarily testosterone and dihydrotestosterone (DHT) are mediated by the androgen receptor (AR), a hormone activated transcription factor and that metastatic prostate cancer (PCa) is treatable with some form of androgen blockade. The present inventors also recognize that, although most tumors respond initially, resistance generally develops within two years (reviewed in (1)) and that additional therapies are needed for these patients. The present inventors also recognize that these tumors, identified as castration resistant prostate cancers (CRPC) may continue to be AR dependent; that typically, AR expression is increased, and a subset of AR responsive genes including PSA (prostate specific antigen) are re-expressed in the tumors (1). The present inventors appreciate evidence from cell lines, mouse models, and responses of patients to additional anti-hormone therapy that the tumors are AR dependent; for example, the castration resistant C4-2 PCa cell line grows in androgen depleted medium and expresses PSA under these conditions, and depletion of AR using siRNA blocks cell growth and PSA expression (2). Moreover, the present inventors recognize that many of these tumors respond to a second form of anti-hormone therapy such as abiraterone (3;4), which further reduces the levels of androgens, or the new AR antagonist in clinical trials, MDV-3100 (4;5). However, the tumors also develop resistance to treatment. A number of AR dependent mechanisms for the development of CRPC have been proposed. These include AR amplification and mutation, local synthesis of androgens (6), increased cell signaling (7;8) and/or increased coactivator expression (1). The present inventors appreciate that constitutively active AR splice variants lacking their hormone binding domains have been described recently (9-12), that these may play an important role in CRPC, and that none of the treatments targeting the hormone-binding domain will be effective against the constitutively active variants. Hence, treatments that target other functional regions of AR or eliminate AR expression are urgently needed.

SEQ ID NO: 1 represents the human AR, transcript variant 1, mRNA.

(SEQ ID NO: 1)
CGAGATCCCGGGGAGCCAGCTTGCTGGGAGAGCGGGACGGTCCGGAGCAAGCC
CAGAGGCAGAGGAGGCGACAGAGGGAAAAAGGGCCGAGCTAGCCGCTCCAGTGCTGT
ACAGGAGCCGAAGGGACGCACCACGCCAGCCCCAGCCCGGCTCCAGCGACAGCCAAC
GCCTCTTGCAGCGCGGCGGCTTCGAAGCCGCCGCCCGGAGCTGCCCTTTCCTCTTCGGT
GAAGTTTTTAAAAGCTGCTAAAGACTCGGAGGAAGCAAGGAAAGTGCCTGGTAGGACT
GACGGCTGCCTTTGTCCTCCTCCTCTCCACCCCGCCTCCCCCCACCCTGCCTTCCCCCCC
TCCCCCGTCTTCTCTCCCGCAGCTGCCTCAGTCGGCTACTCTCAGCCAACCCCCCTCACC
ACCCTTCTCCCCACCCGCCCCCCCGCCCCCGTCGGCCCAGCGCTGCCAGCCCGAGTTTG
CAGAGAGGTAACTCCCTTTGGCTGCGAGCGGGCGAGCTAGCTGCACATTGCAAAGAAG
GCTCTTAGGAGCCAGGCGACTGGGGAGCGGCTTCAGCACTGCAGCCACGACCCGCCTG
GTTAGGCTGCACGCGGAGAGAACCCTCTGTTTTCCCCCACTCTCTCTCCACCTCCTCCTG
CCTTCCCCACCCCGAGTGCGGAGCCAGAGATCAAAAGATGAAAAGGCAGTCAGGTCTT
CAGTAGCCAAAAAACAAAACAAACAAAAACAAAAAAGCCGAAATAAAAGAAAAAGA
TAATAACTCAGTTCTTATTTGCACCTACTTCAGTGGACACTGAATTTGGAAGGTGGAGG
ATTTTGTTTTTTTCTTTTAAGATCTGGGCATCTTTTGAATCTACCCTTCAAGTATTAAGA
GACAGACTGTGAGCCTAGCAGGGCAGATCTTGTCCACCGTGTGTCTTCTTCTGCACGAG
ACTTTGAGGCTGTCAGAGCGCTTTTTGCGTGGTTGCTCCCGCAAGTTTCCTTCTCTGGAG
CTTCCCGCAGGTGGGCAGCTAGCTGCAGCGACTACCGCATCATCACAGCCTGTTGAACT
CTTCTGAGCAAGAGAAGGGGAGGCGGGGTAAGGGAAGTAGGTGGAAGATTCAGCCAA
GCTCAAGGATGGAAGTGCAGTTAGGGCTGGGAAGGGTCTACCCTCGGCCGCCGTCCAA
GACCTACCGAGGAGCTTTCCAGAATCTGTTCCAGAGCGTGCGCGAAGTGATCCAGAAC
CCGGGCCCCAGGCACCCAGAGGCCGCGAGCGCAGCACCTCCCGGCGCCAGTTTGCTGC
TGCTG [symbol] [symbol] [symbol] [symbol] [symbol] [symbol] [symbol] [symbol] [symbol] [symbol] [symbol] [symbol] [symbol]
GGGTGAGGAT
GGTTCTCCCCAAGCCCATCGTAGAGGCCCCACAGGCTACCTGGTCCTGGATGAGGAAC
AGCAACCTTCACAGCCGCAGTCGGCCCTGGAGTGCCACCCCGAGAGAGGTTGCGTCCC
AGAGCCTGGAGCCGCCGTGGCCGCCAGCAAGGGGCTGCCGCAGCAGCTGCCAGCACCT
CCGGACGAGGATGACTCAGCTGCCCCATCCACGTTGTCCCTGCTGGGCCCCACTTTCCC
CGGCTTAAGCAGCTGCTCCGCTGACCTTAAAGACATCCTGAGCGAGGCCAGCACCATG
CAACTCCTTCAGCAACAGCAGCAGGAAGCAGTATCCGAAGGCAGCAGCAGCGGGAGA
GCGAGGGAGGCCTCGGGGGCTCCCACTTCCTCCAAGGACAATTACTTAGGGGGCACTT
CGACCATTTCTGACAACGCCAAGGAGTTGTGTAAGGCAGTGTCGGTGTCCATGGGCCT
GGGTGTGGAGGCGTTGGAGCATCTGAGTCCAGGGGAACAGCTTCGGGGGGATTGCATG
TACGCCCCACTTTTGGGAGTTCCACCCGCTGTGCGTCCCACTCCTTGTGCCCCATTGGCC
GAATGCAAAGGTTCTCTGCTAGACGACAGCGCAGGCAAGAGCACTGAAGATACTGCTG
AGTATTCCCCTTTCAAGGGAGGTTACACCAAAGGGCTAGAAGGCGAGAGCCTAGGCTG
CTCTGGCAGCGCTGCAGCAGGGAGCTCCGGGACACTTGAACTGCCGTCTACCCTGTCTC
TCTACAAGTCCGGAGCACTGGACGAGGCAGCTGCGTACCAGAGTCGCGACTACTACAA
CTTTCCACTGGCTCTGGCCGGACCGCCGCCCCCTCCGCCGCCTCCCCATCCCCACGCTC
GCATCAAGCTGGAGAACCCGCTGGACTACGGCAGCGCCTGGGCGGCTGCGGCGGCGCA
GTGCCGCTATGGGGACCTGGCGAGCCTGCATGGCGCGGGTGCAGCGGGACCCGGTTCT
GGGTCACCCTCAGCCGCCGCTTCCTCATCCTGGCACACTCTCTTCACAGCCGAAGAAGG
CCAGTTGTATGGACCGTGT [symbol] [symbol] [symbol] [symbol] [symbol] [symbol] [symbol] [symbol] [symbol]
GAGGCGGGAGCTGTAGCCCCCTACGGC
TACACTCGGCCCCCTCAGGGGCTGGCGGGCCAGGAAAGCGACTTCACCGCACCTGATG
TGTGGTACCCTGGCGGCATGGTGAGCAGAGTGCCCTATCCCAGTCCCACTTGTGTCAAA
AGCGAAATGGGCCCCTGGATGGATAGCTACTCCGGACCTTACGGGGACATGCGTTTGG
AGACTGCCAGGGACCATGTTTTGCCCATTGACTATTACTTTCCACCCCAGAAGACCTGC
CTGATCTGTGGAGATGAAGCTTCTGGGTGTCACTATGGAGCTCTCACATGTGGAAGCTG
CAAGGTCTTCTTCAAAAGAGCCGCTGAAGGGAAACAGAAGTACCTGTGCGCCAGCAGA
AATGATTGCACTATTGATAAATTCCGAAGGAAAAATTGTCCATCTTGTCGTCTTCGGAA
ATGTTATGAAGCAGGGATGACTCTGGGAGCCCGGAAGCTGAAGAAACTTGGTAATCT
GAAACTACAGGAGGAAGGAGAGGCTTCCAGCACCACCAGCCCCACTGAGGAGACAAC
CCAGAAGCTGACAGTGTCACACATTGAAGGCTATGAATGTCAGCCCATCTTTCTGAATG
TCCTGGAAGCCATTGAGCCAGGTGTAGTGTGTGCTGGACACGACAACAACCAGCCCGA
CTCCTTTGCAGCCTTGCTCTCTAGCCTCAATGAACTGGGAGAGAGACAGCTTGTACACG
TGGTCAAGTGGGCCAAGGCCTTGCCTGGCTTCCGCAACTTACACGTGGACGACCAGAT
GGCTGTCATTCAGTACTCCTGGATGGGGCTCATGGTGTTTGCCATGGGCTGGCGATCCT
TCACCAATGTCAACTCCAGGATGCTCTACTTCGCCCCTGATCTGGTTTTCAATGAGTAC
CGCATGCACAAGTCCCGGATGTACAGCCAGTGTGTCCGAATGAGGCACCTCTCTCAAG
AGTTTGGATGGCTCCAAATCACCCCCCAGGAATTCCTGTGCATGAAAGCACTGCTACTC
TTCAGCATTATTCCAGTGGATGGGCTGAAAAATCAAAAATTCTTTGATGAACTTCGAAT
GAACTACATCAAGGAACTCGATCGTATCATTGCATGCAAAAGAAAAAATCCCACATCC
TGCTCAAGACGCTTCTACCAGCTCACCAAGCTCCTGGACTCCGTGCAGCCTATTGCGAG
AGAGCTGCATCAGTTCACTTTTGACCTGCTAATCAAGTCACACATGGTGAGCGTGGACT
TTCCGGAAATGATGGCAGAGATCATCTCTGTGCAAGTGCCCAAGATCCTTTCTGGGAAA
GTCAAGCCCATCTATTTCCACACCCAGTGAAGCATTGGAAACCCTATTTCCCCACCCCA
GCTCATGCCCCCTTTCAGATGTCTTCTGCCTGTTATAACTCTGCACTACTCCTCTGCAGT
GCCTTGGGGAATTTCCTCTATTGATGTACAGTCTGTCATGAACATGTTCCTGAATTCTAT
TTGCTGGGCTTTTTTTTTCTCTTTCTCTCCTTTCTTTTTCTTCTTCCCTCCCTATCTAACCC
TCCCATGGCACCTTCAGACTTTGCTTCCCATTGTGGCTCCTATCTGTGTTTTGAATGGTG
TTGTATGCCTTTAAATCTGTGATGATCCTCATATGGCCCAGTGTCAAGTTGTGCTTGTTT
ACAGCACTACTCTGTGCCAGCCACACAAACGTTTACTTATCTTATGCCACGGGAAGTTT
AGAGAGCTAAGATTATCTGGGGAAATCAAAACAAAAACAAGCAAAC

The four double-underlined regions in SEQ ID NO: 1 represent the target sites, polyQ and polyG tracks are represented by bold italics, and the bold underline region represents the end of exon 3.

The present inventors appreciate that ideal treatment for CRPC would specifically target AR activity and/or AR expression with little or no effect on other proteins.

The present inventors recognize that, although AR is expressed in a number of other tissues, AR blockade is not associated with severe side effects, thereby providing a better opportunity than chemotherapy in this population of elderly patients because, referring to experience with androgen deprivation therapies as well as studies of patients with complete androgen insensitivity syndrome (mutated, inactive AR) (13). In order to function as a transcriptional activator, AR must localize to the nucleus, bind to specific DNA binding sites, and recruit a series of coactivator complexes that modify histones and other proteins opening up the chromatin and facilitating recruitment of additional proteins required to induce transcription. The present inventors recognize that unlike most steroid receptors, the strongest transactivation domain in AR is in the amino-terminus rather than in the hormone-binding domain.

The present inventors also recognize that the p160 coactivators (SRC-1, SRC-2, and SRC-3) are important for AR function (2;14;15); that the p160 coactivator, SRC-1, interacts with the amino-terminus of AR through its carboxyl terminal glutamine rich region (16;17); that the homologous regions in the other family members likely also interact with AR; and that fragments of this glutamine-rich region are sufficient to inhibit AR activity. However, the size (100 amino acids) precludes cellular uptake without modification.

The present inventors appreciate that there are a number of strategies for targeting a protein interaction domain. For example, it may be possible to find a small molecule that inhibits the protein/protein interaction; this would require a high throughput assay and extensive screening followed by optimization of the compound for clinical applications likely requiring several years of effort. An alternative would be to prepare a cell permeable peptide derivative, but the quantities needed for clinical applications likely would be impractical. Thus, the present invention includes embodiments in which a plasmid Lipoplex combination is used to deliver an expression plasmid that will produce the peptide that inhibits AR action.

In one embodiment of the present disclosure, the delivery vehicle comprises DNA encapsulated in cationic bilamellar invaginated vesicles (BIV)(18;19).

In a preferred embodiment, 200-450 nm BIV are prepared from cholesterol and biodegradable 1,2-dioleoyl-3-trimethyl-ammonio-propane (DOTAP).

In one embodiment, the positive charge is reversibly masked (rm) by the addition of a neutral small molecular weight lipid such as dodecyl-βmaltopyranoside, which prevents initial non-specific uptake (20).

The present inventors recognize that delivery vehicle BIV/plasmid lipoplex can penetrate tumor capillaries and are taken up by fusion, minimizing degradation of DNA (21); that they give higher levels of gene expression when injected in mice than other methods of delivery (22), and that this technology can be used to express transgenes in humans in the target tissue without major toxicity (23, 24).

The present invention also includes an approach for inactivating AR by inhibiting its expression.

The present invention includes AR bi-functional shRNA plasmid, which minimizes off-target effects and optimizes the inhibition of expression of proteins (25, 26).

In one embodiment, the plasmid can be delivered in vivo using BIV delivery vehicle. Expression of the shRNAs is driven by Polymerase II, permitting transcription of multiple shRNAs as a single transcript that is then processed to produce the individual shRNAs (26).

In a preferred embodiment, the AR bi-functional shRNA vectors contain two stem loop structures and a miR30-scaffold and yield two shRNAs. One, which contains a perfect base pair match, is designed to reduce overall expression of mRNA through endonucleolytic cleavage and the other with one or two mismatches to inhibit translation of the target gene and facilitate p-body sequestration and mRNA degradation.

The present invention includes technology to reduce expression of full length AR as well as AR variants. The present inventors recognize that stably transfected inducible AR shRNA in C4-2 cells blocks tumor growth and, in some cases causes xenograft tumor regression (27); and that atelocollagen-mediated systemic AR siRNA administration inhibits the growth of 22RV1 xenografts (28). The present disclosure includes approaches to successfully reduce AR signaling.

The present invention includes embodiments in which combination of reducing AR expression and blocking the AR coactivator binding effectively eliminates AR signaling. This approach provides a treatment for prostate cancer.

The present invention discloses an optimized expression vector for SRCdp and a bi-functional shRNA targeted to eliminate both full length and splice variants of AR (bi-sh-AR).

The present inventors have identified a region of SRC-1 that inhibits AR function when expressed in PCa cells and is distributed throughout the cell. The present disclosure includes an embodiment in which adding a nuclear localization signal to the fragment increases nuclear localization and enhances the potency of the peptide. Thus in one embodiment of the present disclosure, SRCdp also contains a nuclear localization signal with increased potency and efficacy.

Without limiting the present invention, in one embodiment, the final construct is inserted into a pUMVC3 vector (26) for the in vivo applications.

In particular embodiments, the present inventors optimize shRNAs that target AR by utilizing a series of in silico approaches.

In certain embodiments, the present inventors restrict the choice of sequences to the regions common to full length AR and the known AR splice variants (corresponding to approximately amino acids 1-610). One way to identify the optimal shRNAs to prepare the bi-sh-AR for in vivo studies is to evaluate efficacy of knock down of full length AR and of AR splice variants, effects on gene expression, and AR dependent cell growth.

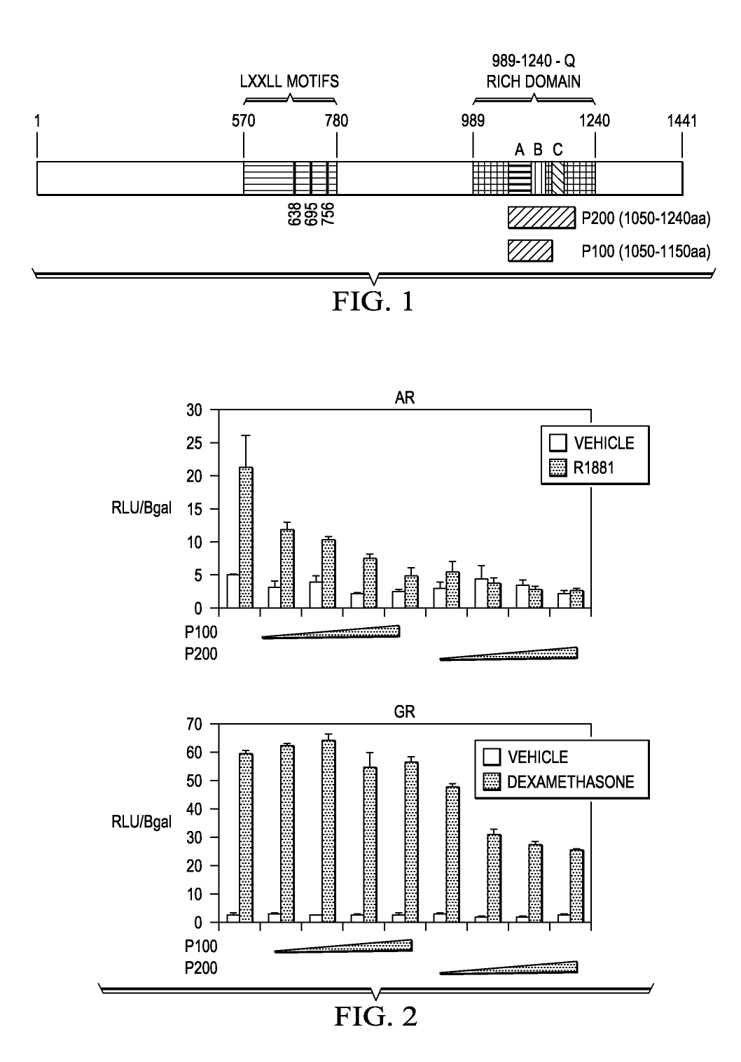

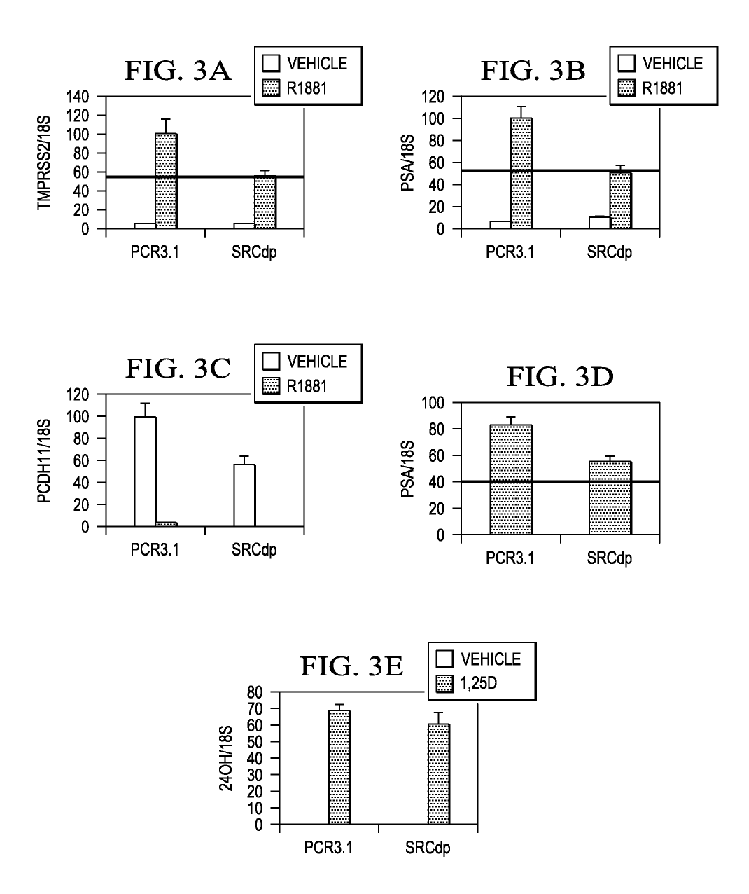

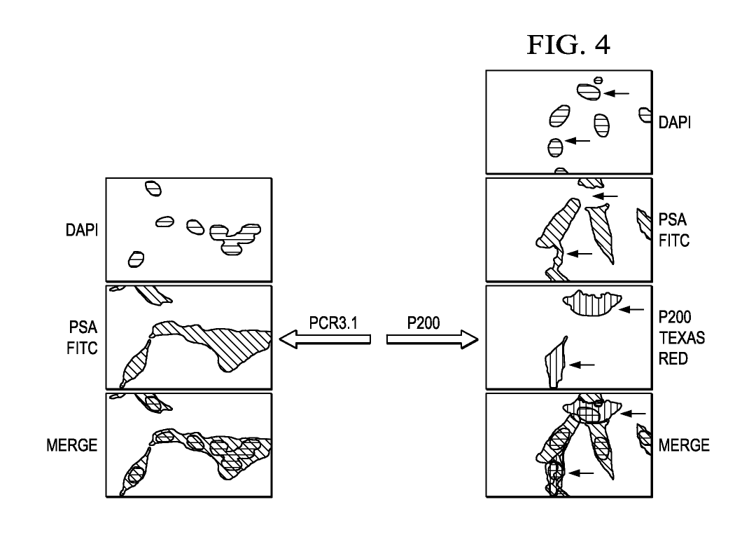

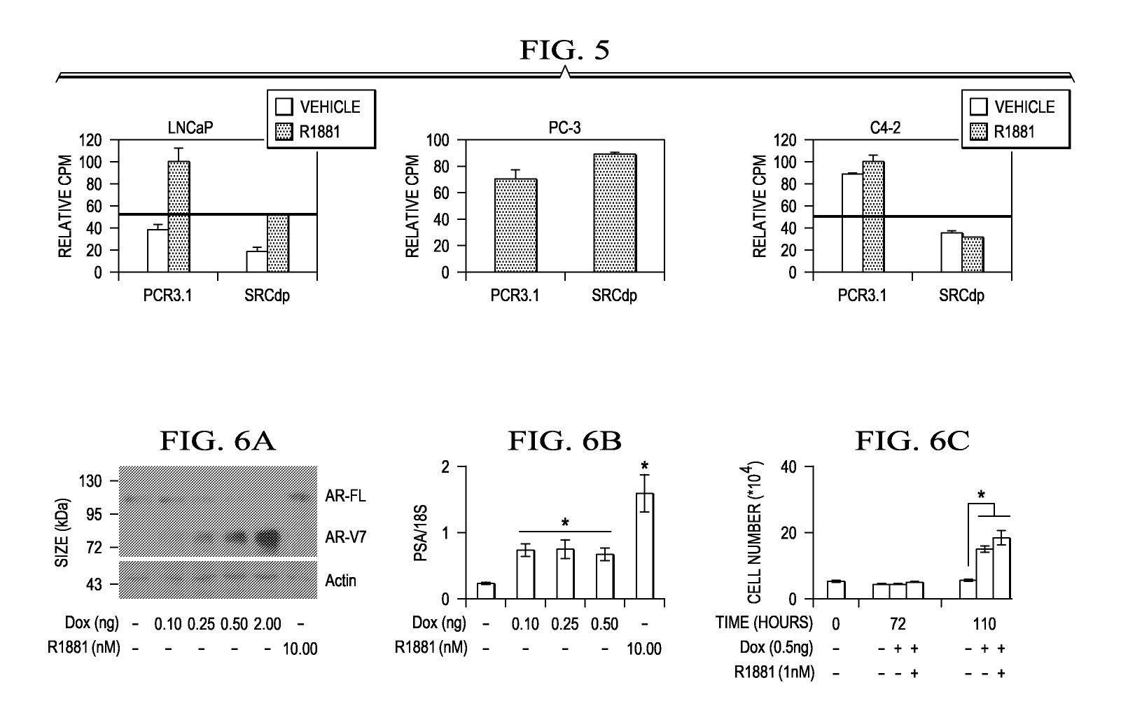

A way to test the efficacy of pUMVC3SRCdp and bi-sh-AR in inhibiting LNCaP xenograft tumor growth is to first, determine the maximum tolerated dose (MTD) of DNA encapsulated in BIV in mice: Male scid mice are injected subcutaneously with a mixture of matrigel and LNCaP cells and tumors allowed to develop for approximately two weeks. Mice are injected IV with optimal doses of BIV containing pUMVC3SRCdp or bi-sh-AR or empty BIV bi-weekly, tumor growth is monitored, and tumors are collected after 6 weeks of treatment. Tumors are characterized for expression of flag SRCdp or for depletion of AR and changes in target genes, changes in Ki67, and apoptosis (TUNEL). Identification of a fragment of SRC-1 that inhibits AR transcriptional activation and cell growth. The importance of the amino-terminal domain of AR in AR function was demonstrated many years ago by evaluating the function of AR lacking its amino-terminus in cell based studies. That the interaction of the amino-terminus of AR with limiting proteins (presumably coactivators) is required for AR dependent tumor growth has been shown in mouse xenograft studies. Overexpression of the amino-terminal portion of AR in androgen dependent LNCaP cells strongly inhibited both cell and xenograft tumor growth (29). Moreover, the role of SRC-1, specifically, in prostate growth was demonstrated in studies of SRC-1 knock-out mice (30). Androgen dependent induction of prostate growth was reduced in these mice compared to control mice. That the role of SRC-1 in cell growth is receptor dependent was shown by our studies showing that depletion of SRC-1 reduced growth of androgen dependent LNCaP cells and CRPC C4-2 cells, but had no effect on the growth of AR negative PC-3 cells (2). FIG. 1 shows the structure of SRC-1 and the location of the peptides derived from SRC-1. The inventors show that these interact with AR, but not with a related receptor, the progesterone receptor. Overexpression of SRC derived peptides (SRCdp) inhibits AR activity measured using an AR responsive reporter, but does not inhibit glucocorticoid receptor (GR) activity at concentrations sufficient to block AR activity (FIG. 2). Moreover, SRCdp (P100 and P200) inhibit induction of target genes including PSA and TMPRSS2, but not the repression of PCDH11, a growth stimulating Wnt family member (FIGS. 3A-3E). The androgen regulated TMPRSS2 gene contains the promoter for the TMPRSS2:ETS factor oncogenic translocation found in the majority of PCa (31) and thus is of particular relevance. Panel C (FIGS. 3A-3E) shows the inhibition of AR dependent, but hormone independent expression of PSA in castration resistant C4-2 cells. Panel E shows that induction of 24OHase by the vitamin D receptor is unaffected by the expression of the peptide. Note that these transient transfections are approximately 50% efficient, so a line has been drawn to indicate the level of residual activity expected at 50% transfection efficiency. An analysis of individual cells for levels of PSA expression and correlation with SRCdp expression, shows a much greater reduction in PSA expression in successfully transfected cells (FIG. 4) although a small number of cells expressing SRCdP retain some PSA expression. Importantly, SRCdp inhibits both LNCaP and C4-2 cell growth with no effect on the growth of AR negative PC-3 cells (FIG. 5). Thus, expression of SRCdp provides a novel means to block AR dependent gene transcription and cell growth. Although our intention in this pilot is to test it as a monotherapy, it clearly could be used in combination with conventional androgen blockade methods or with bi-sh-AR.

The inventors have a number of cell models, which can be used in vitro and as xenografts to test efficacy of the SRCdp, and the candidate bi-sh-ARs. SRCdp efficacy will be tested using parental LNCaP cells, the CRPC C4-2 cells, and our newly developed tetracycline inducible ARV7 LNCaP cell line. This line inducibly expresses the V7 (also termed AR3) variant (10;11), whose expression has been demonstrated by western blotting, immunohistochemistry, and RT-qPCR in many CRPC tumors (10;11) although it is not expressed in C4-2 cells. The present inventors regulate expression as a function of dose (FIG. 6A), demonstrate V7 dependent PSA expression (FIG. 6B) and cell growth (FIG. 6C). In androgen depleted conditions, full length AR is not functional and the activity is V7 dependent. Because natural tumors that express V7 typically express full length AR as well, these mimic typical expression patterns. These cells as well as an additional cell line, which endogenously expresses a wider range of variants (22RV1) (10, 11) will be used to test efficacy of the initial bi-sh-AR constructs.

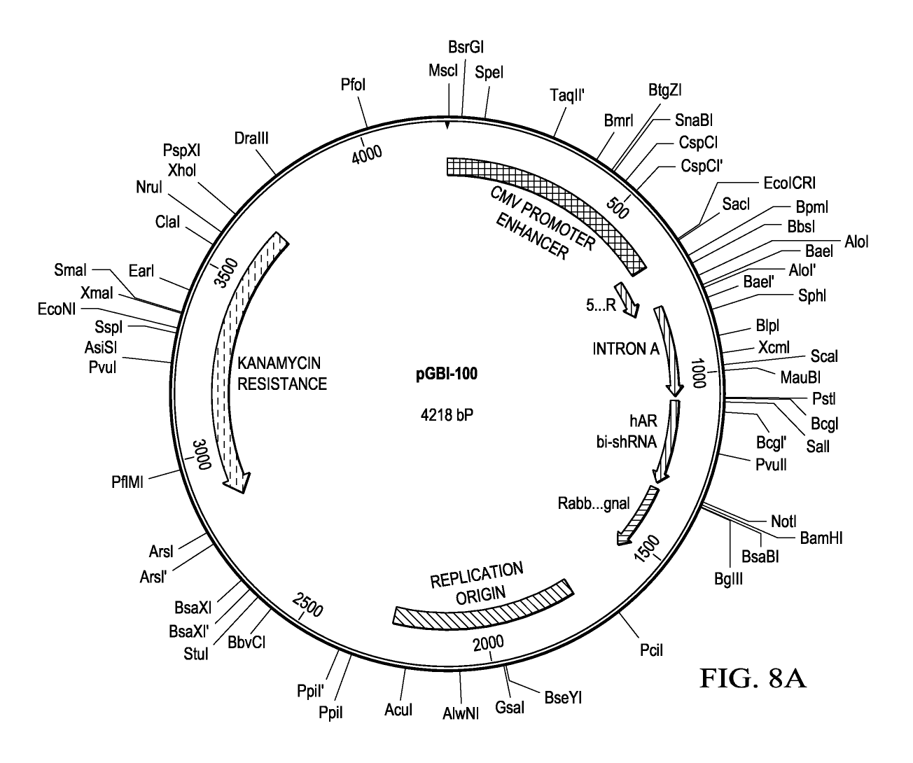

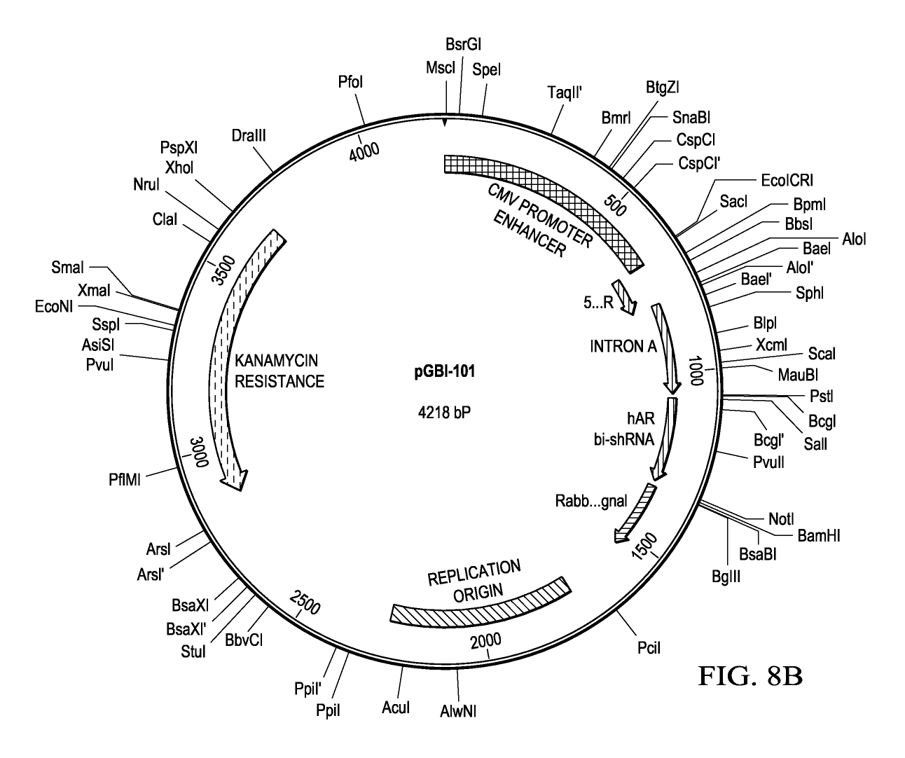

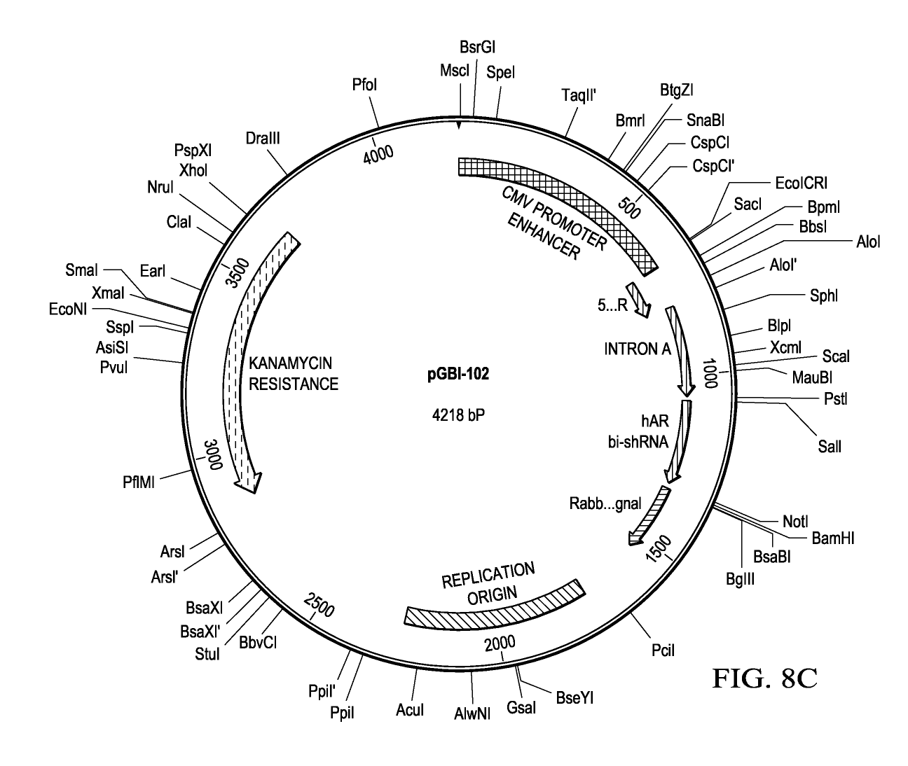

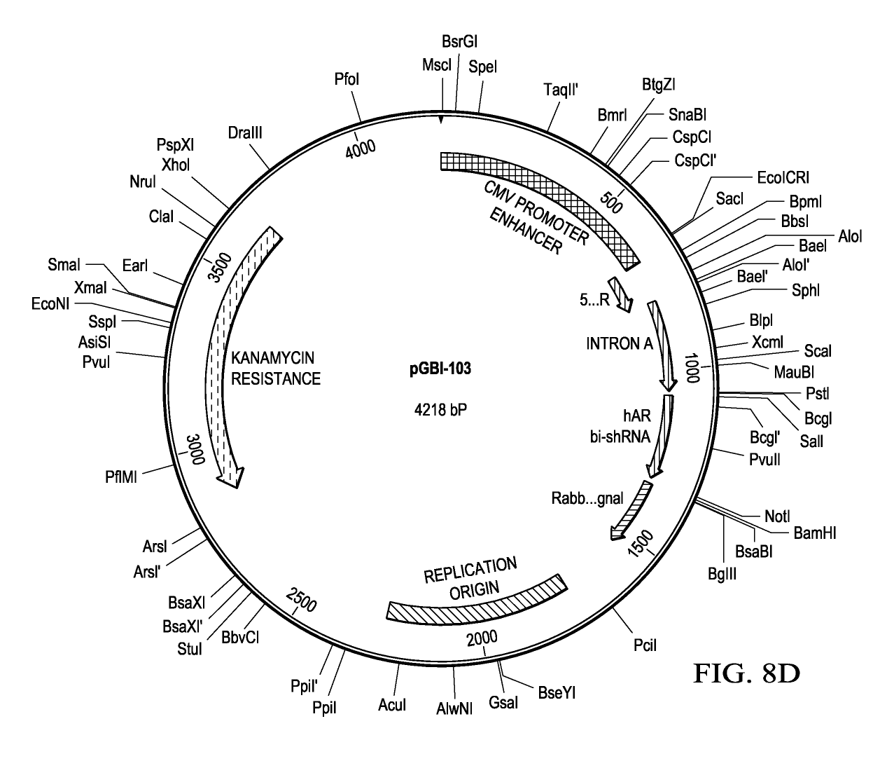

The different candidate bi-sh-AR constructs used in studies conducted in the present invention are shown in Table 1 and the corresponding plasmid maps are shown in FIGS. 8A-8D.

TABLE 1
Candidate bi-sh-Ar constructs.

| | Target Site/Nucleotide | | |
|---|---|---|---|
| Constructs | No. | Sense Sequence | Antisense Sequence |
| hAR1 | CAGAAATGATTGCAC | 5′CAGAAATGATTGC | 5′AATAGTGCAATCA |
| | TATT/ (SEQ ID NO: 2) | ACTATT 3′ (SEQ ID | TTTCTG 3′ (SEQ ID |
| | 2909-2927 | NO: 2) | NO: 3) |
| hAR2 | CAGCCTGTTGAACT | 5′CAGCCTGTTGAACT | 5′AGAAGAGTTCAAC |
| | CTTCT/ (SEQ ID NO: 4) | CTTCT 3′ (SEQ ID | AGGCTG 3′ (SEQ ID |
| | 1037-1055 | NO: 4) | NO: 5) |
| hAR3 | ACGAGGCAGCTGCG | 5′ACGAGGCAGCTGC | 5′TGGTACGCAGCTG |
| | TACCA/ (SEQ ID NO: 6) | GTACCA 3′ (SEQ ID | CCTCGT 3′ (SEQ ID |
| | 2176-2194 | NO: 6) | NO: 7) |
| hAR4 | GCAGGAAGCAGTAT | 5′GCAGGAAGCAGTA | 5′TTCGGATACTGCTT |
| | CCGAA/ (SEQ ID NO: 8) | TCCGAA 3′ (SEQ ID | CCTGC 3′ (SEQ ID |
| | 1709-1727 | NO: 8) | NO: 9) |

The complete sequences corresponding to the different candidate bi-sh-AR constructs shown in Table 1, are presented herein below:

bi-shRNA-hAR1 (pGBI-100)

(SEQ ID NO: 10)
TCGACTGCTGTTGAAGTGAGCGCCCAGAAATGATTGCACTATTTAGTGAAGCCACAGA
TGTAAATAGTGCAATCATTTCTGGTTGCCTACTGCCTCGGAAGCA [symbol] [symbol] [symbol]
GCTGTTGAAGTGAGCGCCCAGAAATGTCAGCACCATTTAGTGAAGCCACAGATGT
AAATAGTGCAATCATTTCTGGT [symbol] AATAAAGGATCTTTTAT
TTTCATTGGC

bi-shRNA-hAR2 (pGBI-101)

(SEQ ID NO: 11)
TCGACTGCTGTTGAAGTGAGCGCCCAGCCTGTTGAACTCTTCTTAGTGAAGCCACAGAT
GTAAGAAGAGTTCAACAGGCTGGTTGCCTACTGCCTCGGAAGC [symbol] [symbol] [symbol]
GCTGTTGAAGTGAGCGCCCAGCCTGTATCACTATTCTTAGTGAAGCCACAGATGTAA
GAAGAGTTCAACAGGCTGGT [symbol] AATAAAGGATCTTTTATT
TTCATTGGC

bi-shRNA-hAR3 (pGBI-102)

(SEQ ID NO: 12)
TCGACTGCTGTTGAAGTGAGCGCCACGAGGCAGCTGCGTACCATAGTGAAGCCACAGA
TGTATGGTACGCAGCTGCCTCGTGTTGCCTACTGCCTCGGAAGC [symbol] [symbol] [symbol]
GCTGTTGAAGTGAGCGCCACGAGGCACTAGCGTATCATAGTGAAGCCACAGATGT
ATGGTACGCAGCTGCCTCGTGT [symbol] AATAAAGGATCTTTTA
TTTTCATTGGC

bi-shRNA-hAR4 (pGBI-103)

(SEQ ID NO: 13)
TCGACTGCTGTTGAAGTGAGCGCCGCAGGAAGCAGTATCCGAATAGTGAAGCCACAGA
TGTATTCGGATACTGCTTCCTGCGTTGCCTACTGCCTCGGAAGC [symbol] [symbol] [symbol]
GCTGTTGAAGTGAGCGCCGCAGGAAGTTCTATCAGAATAGTGAAGCCACAGATGTA
TTCGGATACTGCTTCCTGCGT [symbol] AATAAAGGATCTTTTATT
TTCATTGGC

The present invention identifies a fragment of SRC-1 that inhibits AR transcriptional activation and cell growth. The present inventors recognize the importance of the amino-terminal domain of AR in AR function as demonstrated by evaluating the function of AR lacking its amino-terminus in cell-based studies.

The present inventors also recognize that the interaction of the amino-terminus of AR with limiting proteins (presumably coactivators) is required for AR dependent tumor growth, based on xenograft studies in mouse.

The present inventors appreciate that overexpression of the amino-terminal portion of AR in androgen dependent LNCaP cells strongly inhibits both cell and xenograft tumor growth (29); and that the role of SRC-1, specifically, in prostate growth is demonstrated in studies of SRC-1 knock-out mice (30). In these mice, Androgen dependent induction of prostate growth is reduced compared to control mice.

The present inventors show that the role of SRC-1 in cell growth is receptor dependent in studies showing that depletion of SRC-1 reduces growth of androgen dependent LNCaP cells and CRPC C4-2 cells, but has no effect on the growth of AR negative PC-3 cells (2).

FIG. 1 shows the structure of SRC-1 and the location of the peptides derived from SRC-1. These peptides interact with AR but not with a related receptor, the progesterone receptor. Overexpression of SRC derived peptides (SRCdp) inhibits AR activity measured using an AR responsive reporter, but does not inhibit glucocorticoid receptor (GR) activity at concentrations sufficient to block AR activity (FIG. 2).

SRCdp (P100 and P200) inhibit induction of target genes including PSA and TMPRSS2, but not the repression of PCDH11, a growth stimulating Wnt family member (FIG. 3). The androgen regulated TMPRSS2 gene contains the promoter for the TMPRSS2:ETS factor oncogenic translocation found in the majority of PCa (31), and the present inventors recognize its particular relevance.

Panel C (FIGS. 3A-3E) shows the inhibition of AR dependent but hormone independent expression of PSA in castration-resistant C4-2 cells.

Panel E shows that induction of 24OHase by the vitamin D receptor is unaffected by the expression of the peptide. The present inventor note that these transient transfections are approximately 50% efficient; so, a line has been drawn to indicate the level of residual activity expected at 50% transfection efficiency.

An analysis of individual cells for levels of PSA expression and correlation with SRCdp expression, shows a much greater reduction in PSA expression in successfully transfected cells (FIG. 4) although a small number of cells expressing SRCdP appear to retain some PSA expression.

Importantly, SRCdp inhibits both LNCaP and C4-2 cell growth with no effect on the growth of AR negative PC-3 cells (FIG. 5). Thus, expression of SRCdp provides means to block AR dependent gene transcription and cell growth.

In a non-limiting embodiment SRCdp is employed as a monotherapy; in another embodiment, SRCdp is used in combination with conventional androgen blockade methods and, or, in combination with bi-sh-AR.

The present inventors employ a number of cell models, which can be used in vitro and as xenografts to test efficacy of the SRCdp, and bi-sh-ARs. One way to show SRCdp efficacy is to use parental LNCaP cells, CRPC C4-2 cells, or tetracycline inducible ARV7 LNCaP cell lines, which expresses the V7 (also termed AR3) variant (10;11), whose expression has been demonstrated by western blotting, immunohistochemistry, and RT-qPCR in many CRPC tumors (10;11) although it is not expressed in C4-2 cells. Expression can be regulated as a function of dose (FIG. 6A), demonstrate V7 dependent PSA expression (6B) and cell growth (6C). The present inventors recognize that in androgen depleted conditions, full length AR is not functional and the activity is V7 dependent. Because natural tumors that express V7 typically express full length AR as well, these mimic typical expression patterns. One way to test efficacy of the initial bi-sh-AR constructs is to use these cells as well as an additional cell line, which endogenously expresses a wider range of variants (22RV1) (10;11).

The present inventor develop and perform in vitro tests of an optimized expression vector for SRCdp and a bi-functional shRNA targeted to eliminate both full length and splice variants of AR (bi-sh-AR).

The present invention includes two fragments of SRC-1, one containing approximately 200 amino acids (amino acids 1050-1240) and one containing 100 amino acids (1050-1150) are capable of inhibiting the transcriptional activity of AR. Although a shorter piece is likely to give fewer side effects, it appears that the 200 amino acid fragment expresses better (or is more stable than the 100 amino acid fragment). One way to determine optimal inhibition is to prepare both with nuclear localization signals.

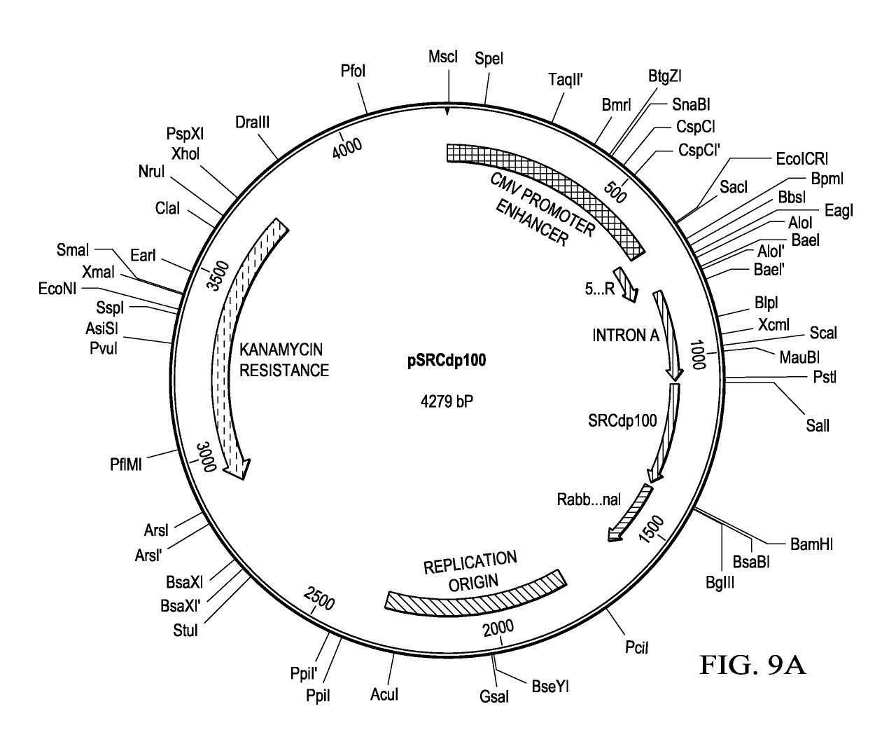

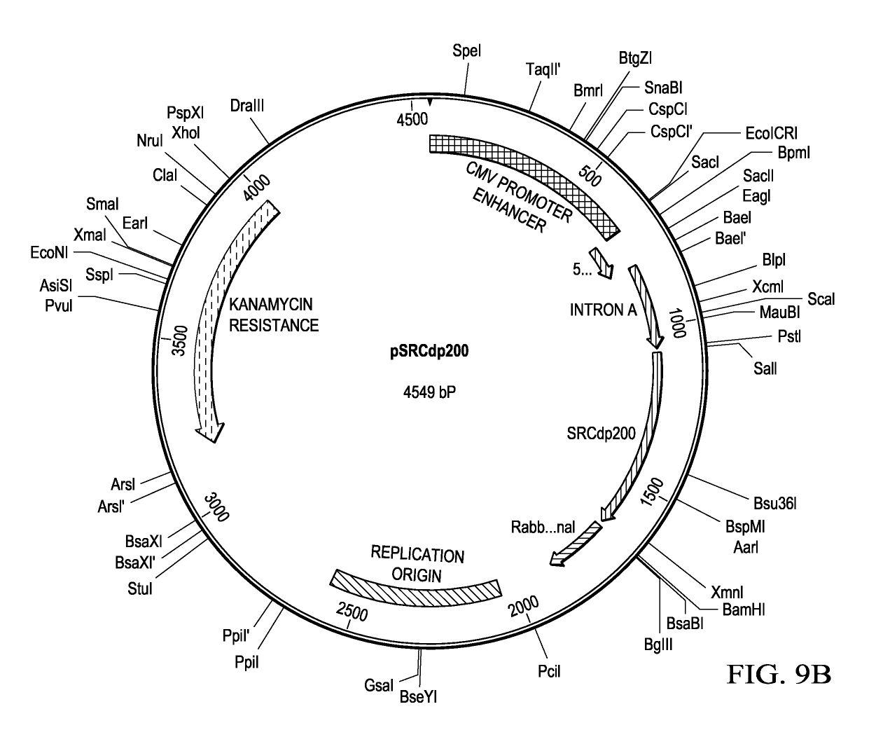

The amino acid and the mRNA sequences corresponding to the two fragments of SRC-1 are presented herein below. The plasmid maps corresponding to the two fragments are shown in FIGS. 9A and 9B.

SRCdp100: Amino acids 1050-1150 of SRC-1

(SEQ ID NO: 14)
MAPNQLRLQLQQRLQGQQQLIHQNRQAILNQFAATAPVGINMRSGMQQQ
ITPQPPLNAQMLAQRQRELYSQQHRQRQLIQQQRAMLMRQQSFGNNLPP
SSGL

SRCdp100 mRNA sequence:

(SEQ ID NO: 15)
ATGGCACCTAACCAGCTTCGACTTCAACTACAGCAGCGATTACAGGGAC
AACAGCAGTTGATACACCAAAATCGGCAAGCTATCTTAAACCAGTTTGC
AGCAACTGCTCCTGTTGGCATCAATATGAGATCAGGCATGCAACAGCAA
ATTACACCTCAGCCACCCCTGAATGCTCAAATGTTGGCACAACGTCAGC
GGGAACTGTACAGTCAACAGCACCGACAGAGGCAGCTAATACAGCAGCA
AAGAGCCATGCTTATGAGGCAGCAAAGCTTTGGGAACAACCTCCCTCCC
TCATCTGGACTATAG

SRCdp200: Amino acids 1050-1240 of SRC-1

(SEQ ID NO: 16)
MAPNQLRLQLQQRLQGQQQLIHQNRQAILNQFAATAPVGINMRSGMQQQITPQPPLNAQM
LAQRQRELYSQQHRQRQLIQQQRAMLMRQQSFGNNLPPSSGLPVQMGNPRLPQGAPQQFP
YPPNYGTNPGTPPASTSPFSQLAANPEASLANRNSMVSRGMTGNIGGQFGTGINPQMQQNVFQY
PGAGMVPQ

SRCdp200 mRNA sequence:

(SEQ ID NO: 17)
ATGGCACCTAACCAGCTTCGACTTCAACTACAGCAGCGATTACAGGGACAACAGCAGT
TGATACACCAAAATCGGCAAGCTATCTTAAACCAGTTTGCAGCAACTGCTCCTGTTGGC
ATCAATATGAGATCAGGCATGCAACAGCAAATTACACCTCAGCCACCCCTGAATGCTC
AAATGTTGGCACAACGTCAGCGGGAACTGTACAGTCAACAGCACCGACAGAGGCAGCT
AATACAGCAGCAAAGAGCCATGCTTATGAGGCAGCAAAGCTTTGGGAACAACCTCCCT
CCCTCATCTGGACTACCAGTTCAAATGGGGAACCCCCGTCTTCCTCAGGGTGCTCCACAGC
AATTCCCCTATCCACCAAACTATGGTACAAATCCAGGAACCCCACCTGCTTCTACCAGCCCGT
TTTCACAACTAGCAGCAAATCCTGAAGCATCCTTGGCCAACCGCAACAGCATGGTGAGCAGA
GGCATGACAGGAAACATAGGAGGACAGTTTGGCACTGGAATCAATCCTCAGATGCAGCAGAA
TGTCTTCCAGTATCCAGGAGCAGGAATGGTTCCCCAATAG

The present invention includes embodiments in which a plasmid with or without the flag epitope is followed by the nuclear localization signal DDLPRRRGRS (SEQ ID NO: 18) and either the 100 amino acid fragment (NLSp100) or the 200 amino acid fragment (NLSp200). One way to compare the efficiency of inhibition of AR transcriptional activity with that of our P200 and P100 constructs is using an AR responsive reporter in HeLa cells. The present disclosure also includes embodiments in which the expression plasmids will be delivered, to e.g., LNCaP cells, by electroporation to increase efficiency beyond lipid based transient transfections.

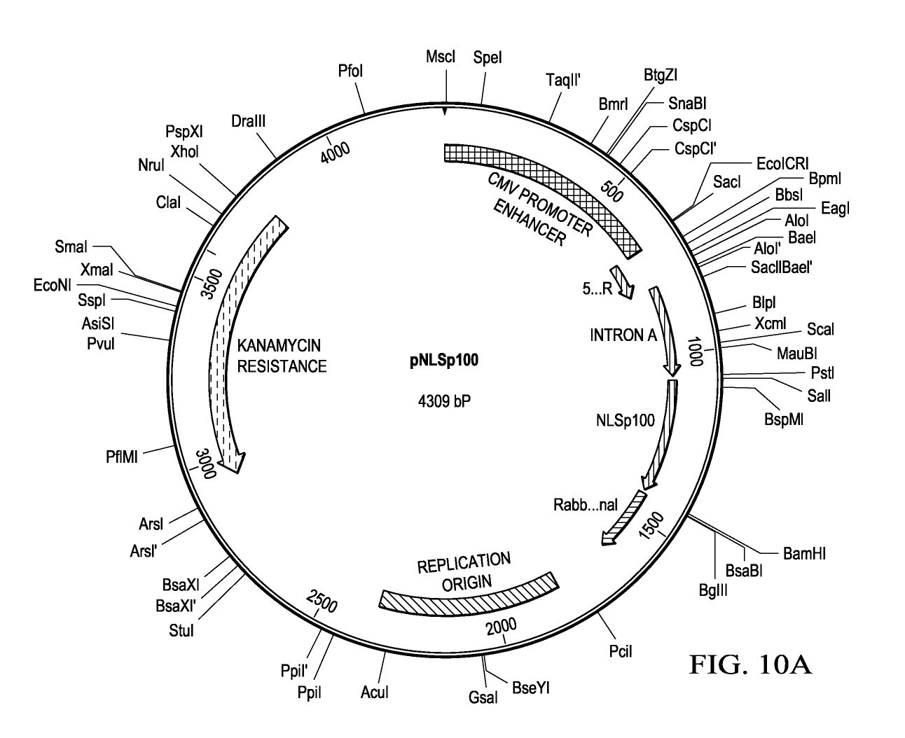

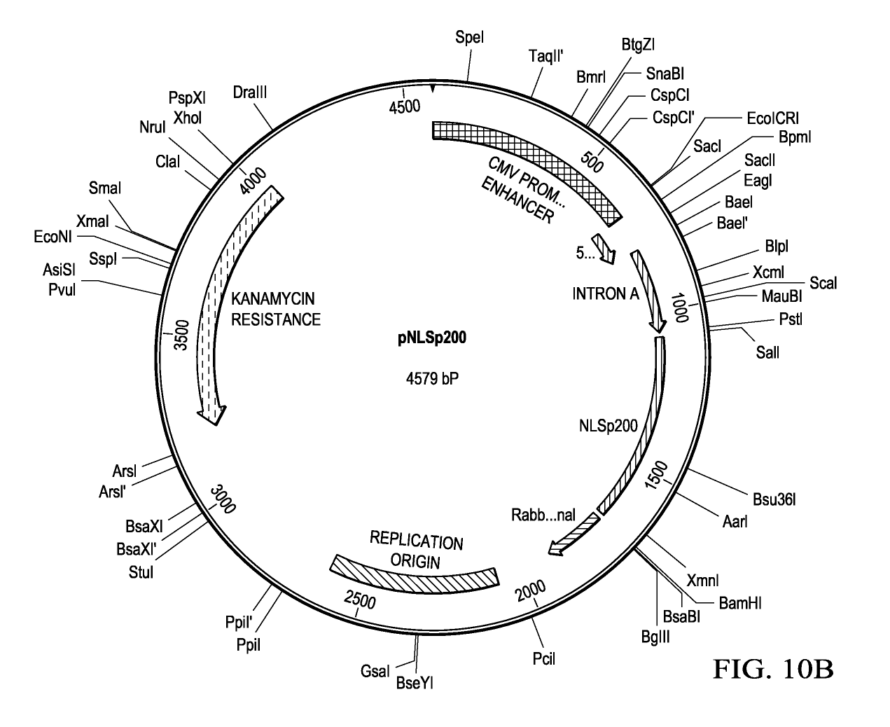

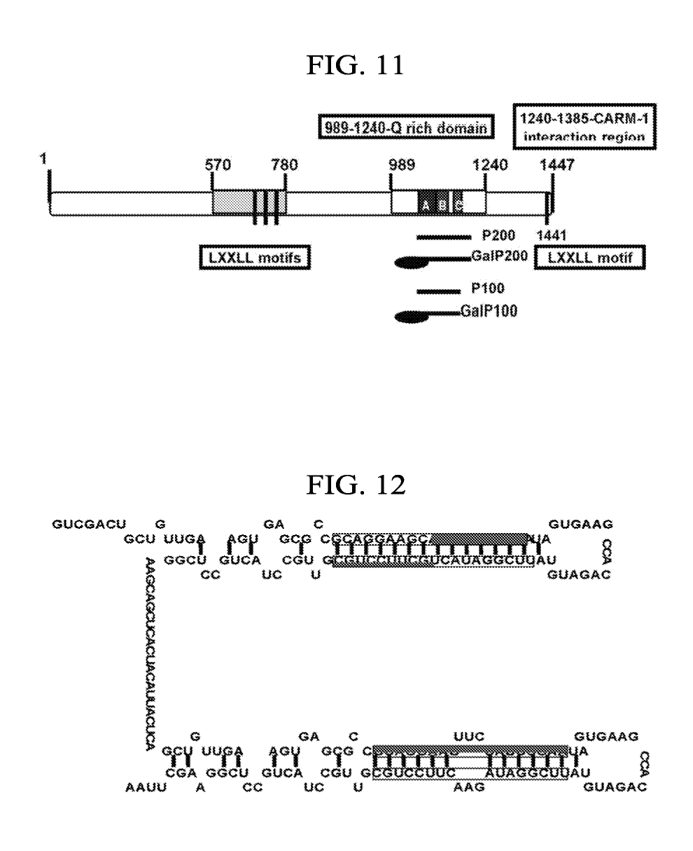

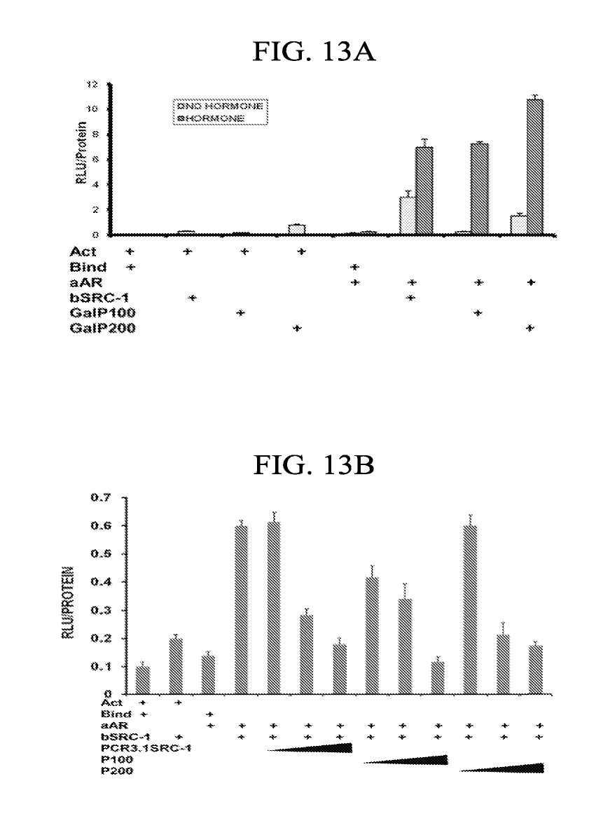

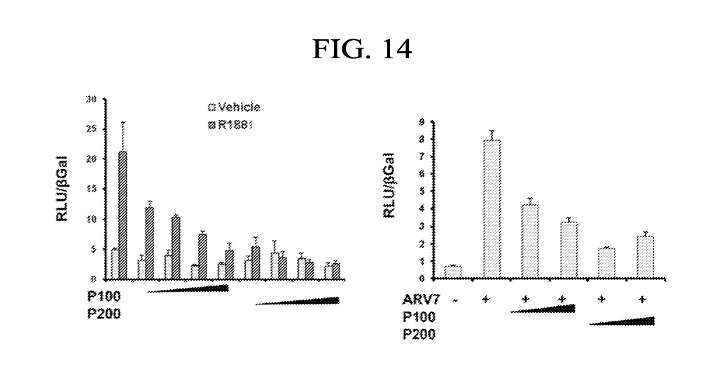

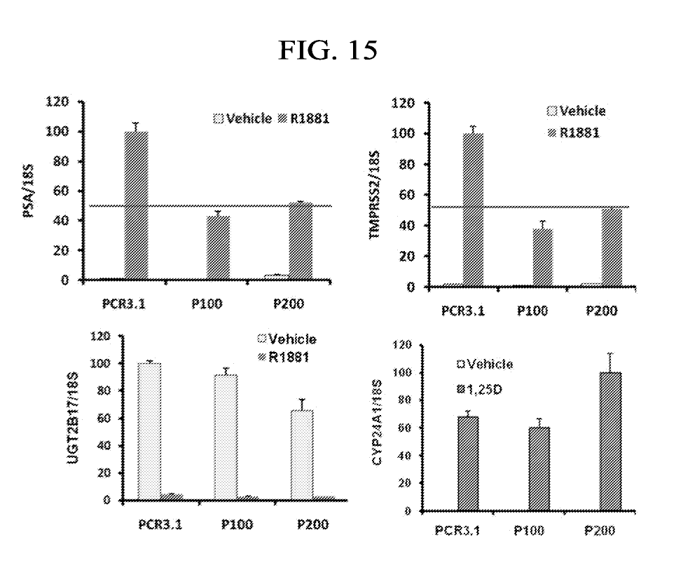

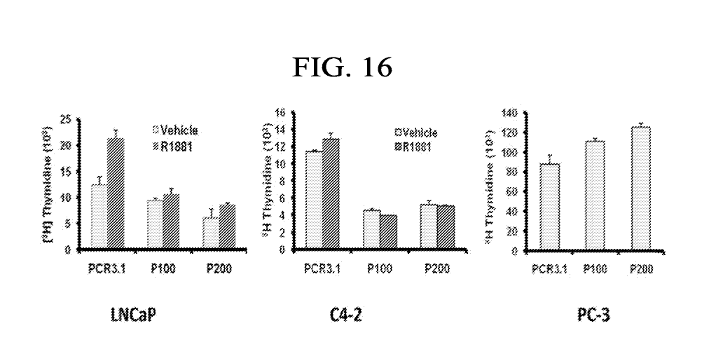

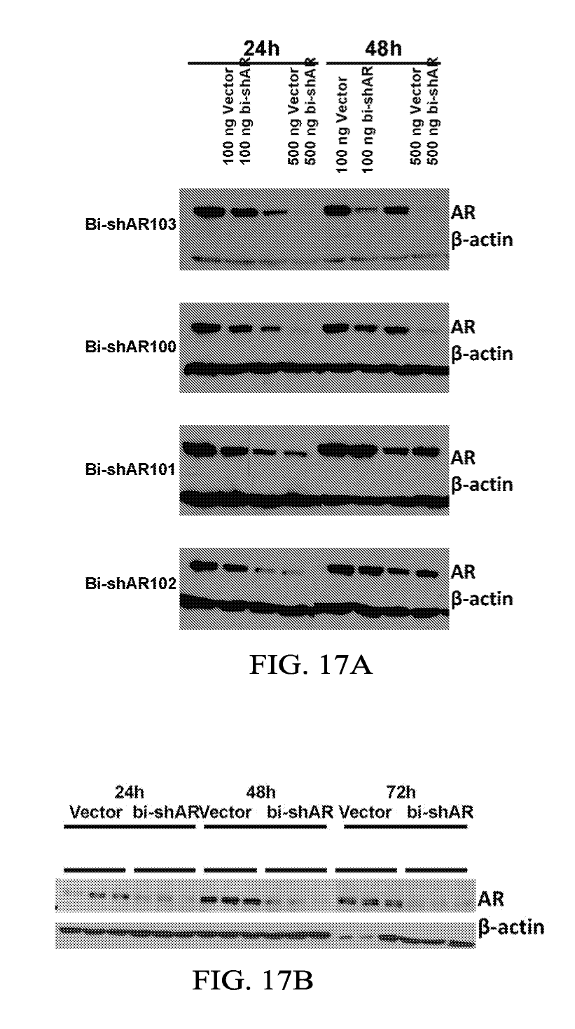

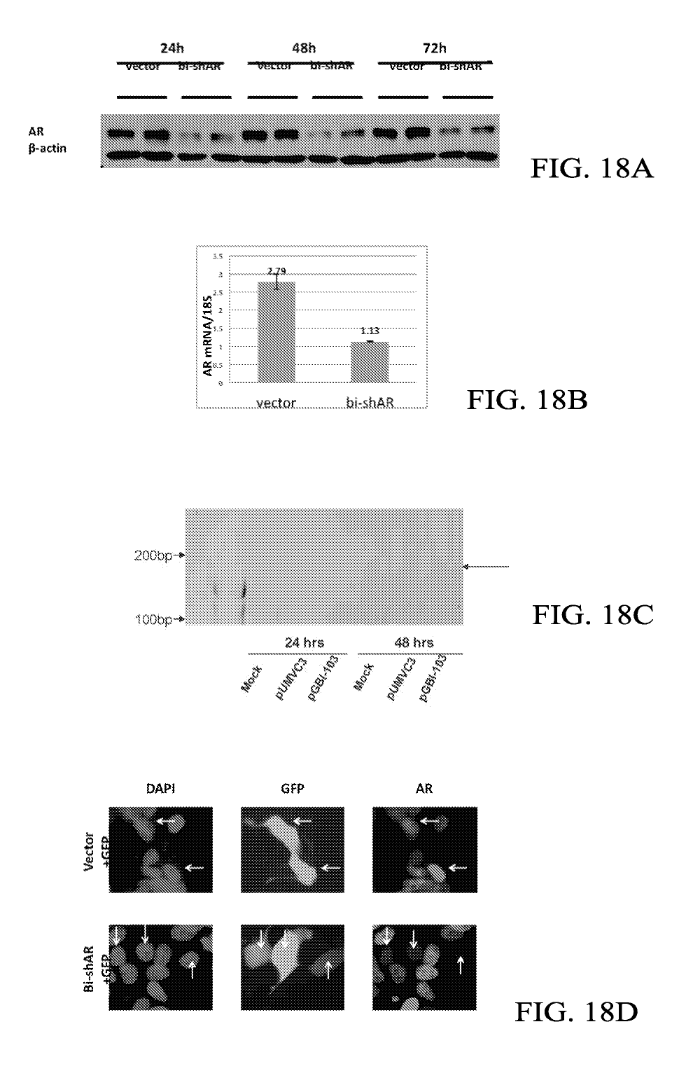

The amino acid and the mRNA sequences corresponding to the 100 amino acid fragment (NLSp100) and the 200 amino acid fragment (NLSp200) are presented herein below. The plasmid maps corresponding to the two fragments are shown in FIGS. 10A and 10B.

pNLSp100 amino acids sequence:

(SEQ ID NO: 19)
MDDLPRRRGRSAPNQLRLQLQQRLQGQQQLIHQNRQAILNQFAATAPVG
INMRSGMQQQITPQPPLNAQMLAQRQRELYSQQHRQRQLIQQQRAMLMR
QQSFGNNLPPSSGL

pNLSp100 mRNA sequence:

(SEQ ID NO: 20)
ATGGACGACCTGCCCAGAAGAAGAGGCAGATCCGCACCTAACCAGCTTC
GACTTCAACTACAGCAGCGATTACAGGGACAACAGCAGTTGATACACCA
AAATCGGCAAGCTATCTTAAACCAGTTTGCAGCAACTGCTCCTGTTGGC
ATCAATATGAGATCAGGCATGCAACAGCAAATTACACCTCAGCCACCCC
TGAATGCTCAAATGTTGGCACAACGTCAGCGGGAACTGTACAGTCAACA
GCACCGACAGAGGCAGCTAATACAGCAGCAAAGAGCCATGCTTATGAGG
CAGCAAAGCTTTGGGAACAACCTCCCTCCCTCATCTGGACTATAG

pNLSp200 amino acids sequence:

(SEQ ID NO: 21)
MDDLPRRRGRSAPNQLRLQLQQRLQGQQQLIHQNRQAILNQFAATAPVGINMRSGMQQQ
ITPQPPLNAQMLAQRQRELYSQQHRQRQLIQQQRAMLMRQQSFGNNLPPSSGLPVQMGNP
RLPQGAPQQFPYPPNYGTNPGTPPASTSPFSQLAANPEASLANRNSMVSRGMTGNIGGQFGTGI
NPQMQQNVFQYPGAGMVPQ

pNLSp200 mRNA sequence:

(SEQ ID NO: 22)
ATGGACGACCTGCCCAGAAGAAGAGGCAGATCCGCACCTAACCAGCTTCGACTTCA
ACTACAGCAGCGATTACAGGGACAACAGCAGTTGATACACCAAAATCGGCAAGCTATC
TTAAACCAGTTTGCAGCAACTGCTCCTGTTGGCATCAATATGAGATCAGGCATGCAACA
GCAAATTACACCTCAGCCACCCCTGAATGCTCAAATGTTGGCACAACGTCAGCGGGAA
CTGTACAGTCAACAGCACCGACAGAGGCAGCTAATACAGCAGCAAAGAGCCATGCTTA
TGAGGCAGCAAAGCTTTGGGAACAACCTCCCTCCCTCATCTGGACTACCAGTTCAAATG
GGGAACCCCCGTCTTCCTCAGGGTGCTCCACAGCAATTCCCCTATCCACCAAACTATGGTAC
AAATCCAGGAACCCCACCTGCTTCTACCAGCCCGTTTTCACAACTAGCAGCAAATCCTGAAGC
ATCCTTGGCCAACCGCAACAGCATGGTGAGCAGAGGCATGACAGGAAACATAGGAGGACAG
TTTGGCACTGGAATCAATCCTCAGATGCAGCAGAATGTCTTCCAGTATCCAGGAGCAGGAATG
GTTCCCCAATAG

One way to measure inhibition of androgen dependent induction of PSA employs quantitative RT-PCR and examines PSA expression, peptide expression, and localization. A way to measure efficiency of electroporation is by parallel electroporation using a β galactosidase reporter followed by staining of the cells with X-Gal to determine the percent of cells successfully electroporated. The present inventors recognize the importance of confirming that the peptides inhibit LNCaP growth without inhibiting PC-3 cell growth. A way to test efficacy of inhibition of AR activity is to use androgen-dependent LAPC-4, androgen dependent VCaP prostate cancer cells, and the 22RV1 cells, which express AR variants.

The present invention discloses shRNA. The inventors recognize the need to eliminate all of the AR and its variants and that the amino-terminus and the DNA binding domain are common to all functional AR forms. In a preferred embodiment, shRNA target sites are located within the region containing exons 1, 2, and 3 (610 amino acids).

The present inventors also recognize that AR has two unusual features in its amino terminus, a variable length poly Gln repeat and a somewhat variable poly Gly region. These regions are eliminated from consideration because of the potential for variability among patients in these areas. An approach to identify further target sites employs algorithms. The inventors recognize that potential target sites are to be further analyzed in respect to its accessibility and with BLAST search for potential “off-target” effect on other human expressed genes.

In a preferred embodiment, the chosen sequence will also deplete mouse AR mRNA and block translation, since this would also serve as a preliminary test for non-tumor effects. The inventors do not expect major toxicity due to any reduction in non-tumor tissues. After the initial screen, the available target sites are inserted into a miR-30a backbone expression construct to generate bi-functional shRNA.

The constructs are tested by transient co-transfection of an AR expression and by testing shRNAs in HeLa cells. Effective shRNAs are then tested against endogenous AR. To test shRNAs, LNCaP cells, LNCaP cells expressing V7 and 22RV1 cells are electroporated with a concentration range of shRNA constructs and cells are harvested after 24, 48, 72, and 96 hours. AR protein is measured by western blotting and comparative knockdown is assessed. AR mRNA is measured and the reduction in androgen or doxycycline dependent induction of the AR target genes PSA and TMPRSS2 can be measured by RT-qPCR.

The present inventors recognize the importance of comparing the effects on cell growth and determining whether depletion of AR induces apoptosis (caspase and Parp-1 cleavage, generation of a sub-G1 peak measured using flow cytometry, and DNA cleavage measured using a Cell Death ELISA) (32;33) or only reduces proliferation (3H thymidine) (34).

The present inventors recognize that a SRC fragment with a nuclear localization signal may be more effective in inhibiting AR activity at lower plasmid levels than a peptide that is broadly distributed within the cell because a SRC fragment with a nuclear localization signal provides higher concentrations in the biologically relevant compartment and nuclear proteins also may be less susceptible to degradation.

In a preferred embodiment, a p100 plasmids will be used that is as effective as the best p200 plasmid, in order to minimize possibilities for any potential off-target effects. Because the interaction between AR and the SRCdp is direct, the present inventors do not expect variability in the ability of the peptide to inhibit AR action between cell lines other than any variation that can be attributed to transfection efficiency.

Nonetheless, the present inventors employ four independent cell lines in case there are unanticipated differences in trafficking or nuclear retention. The present inventors recognize that the possibility of testing against mouse AR at the same time provides an added level of analysis for non-tumor effects but is not absolutely required. On way of directly testing inhibition of expression with little complications due to incomplete transfection is to perform co-transfection studies.

The present inventors recognize that electroporation of LNCaP cells yields very high efficiency for siRNAs (35), plasmid electroporation may be somewhat less efficient. Anti-androgens generally are cytostatic in cell-based assays. The present inventors also recognize that complete elimination of AR may induce apoptosis, although, usually under specific conditions such as serum free conditions. The present inventors appreciate the benefit of testing for apoptosis and, if apoptosis is detected, determining whether it is an off-target effect by measuring effects of the bi-sh-AR in cells such as PC-3 cells, which lack functional AR.

The present inventors recognize the need of testing the efficacy of pUMVC3SRCdp and bi-sh-AR in inhibiting LNCaP xenograft tumor growth.

One way to determine the maximum tolerated dose (MTD) is by injecting male BALB/c mice (5/group) IV with 0, 30, 40, 50, or 60 μg of DNA encapsulated in BIV in a total of 200 ul. Mice are weighed prior to injection and monitored daily by cage side behavior observation and weights. Most side effects are detected within a day, but mice are observed for up to a week. Once the MTD has been identified, 3 scid mice are injected with the optimal dose to confirm that they are not differentially sensitive to the treatment.

Regarding SRCdp and bi-sh-AR on LNCaP tumor growth, one way to determine efficacy is to inject male scid mice subcutaneously on the flank with a mixture of matrigel and 2 million LNCaP cells, and tumors are allowed to develop for approximately two weeks. Mice (15/group) are injected IV with optimal doses of BIV containing pUMVC3SRCdp or bi-sh-AR or empty BIV bi-weekly, animals are weighed weekly, tumor growth are measured with calipers twice a week, and tumors are collected after 6-10 weeks of treatment depending on the rate of control tumor growth (experiment are terminated before 10 weeks if controls tumors reach 1 cm3). Tumors are characterized for weight, expression of flag SRCdp or for depletion of AR as well as changes in AR target genes, changes in Ki67, and apoptosis (TUNEL). Other major organs and, particularly, prostate are collected and reserved for studies. Bio-statistical calculations for animal numbers for the study are based on take rate and variation in the tumor size for LNCaP cells. They are powered to have 80% confidence of seeing a 50% change in growth with a significance level (alpha) of 0.05 using a two-sided two-sample t-test.

Regarding interpretation of results and potential problems, the present inventors recognize that, it is important to test approaches that are successful in blocking tumor growth, in other models and to examine toxicity. The present inventors also recognize importance of testing against CRPC models. One way to conduct testing is to choose LNCaP cells because of the well-characterized androgen and AR dependence of this model.

The present inventors recognize that, if adequate reduction in AR expression or AR signaling is achieved, tumor growth is blocked or even reversed. Based on the specificity of SRCdp for AR action and the lack of a requirement for AR for survival, only minimal toxicity of the plasmids is expected.

In a preferred embodiment, the choice of doses for MTD is 50 ug.

The present inventors recognize that anti-androgen therapies typically inhibit tumor growth but do not cause regression. The present inventors recognize that, in embodiments in which treatment provides better AR blockade, regression occurs.

In a preferred embodiment, two or more approaches of treatment are combined to provide a more effective treatment than either alone.

A particular embodiment comprises a single vector-based product involving both UMVC3SRCdp and bi-sh-AR.

One way to test the bi-sh-AR is to measure the weight of the prostate and test for reduction in AR expression. The present inventors recognize that SRCdp, if delivered in sufficient quantities to mouse prostate, inhibits AR action and reduces prostate size. The core 100 amino acid SRCdp fragment has only 6 amino acid substitutions in mouse compared to human and the 200 amino acid fragment contains 16 substitutions. One way to confirm that mouse AR is inhibited by human SRCdp is to use transfection assays as described in FIG. 2. One way to test AR inhibition is to collect mouse prostate and weigh and measure expression of SRCdp.

Another way to test plasmids and delivery of plasmid is to compare the amount of expression of SRCdp and reduction in AR with that achieved in the cell based studies.

In one embodiment, delivery to prostate tumor is optimized by including small molecules in the BIV that bind to a prostate specific cell-surface molecule such as PSMA. One way to identify short ligands targeted towards different cancer types is to utilize a combinatory library approach (20). The present inventors recognize that this approach is a way to provide further refinement to increase efficiency and specificity if needed.

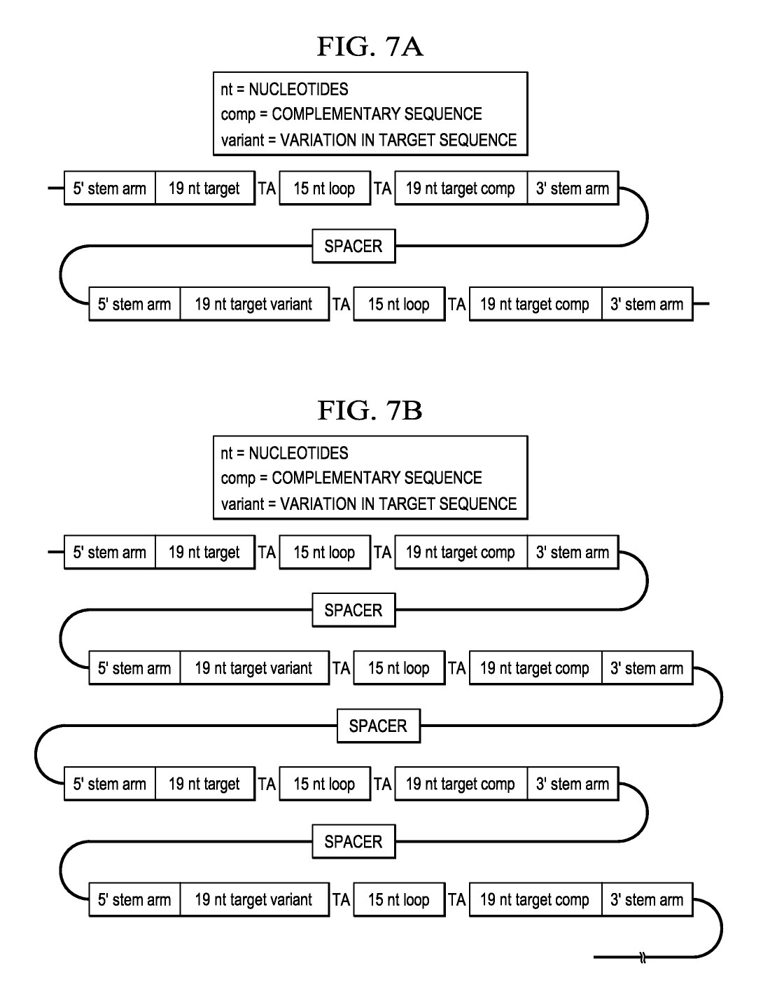

Regarding bi-shRNA, the present inventors have pioneered an unique RNAi platform known as bi-functional shRNA. Conceptually, RNAi can be achieved through shRNA-loaded RISCs to promote cleavage-dependent or cleavage-independent mRNA knockdown. Concomitant expression of both configurations of shRNAs (hence the nomenclature, bi-functional shRNA) has been shown by the present inventors to achieve more effective target gene knockdown at a more rapid onset of silencing (rate of mRNA and protein turnover notwithstanding) with greater durability as compared with siRNA. The basic design of the bi-functional shRNA expression unit comprises two stem-loop shRNA structures; one composed of fully matched passenger and guide strands for cleavage-dependent RISC loading, and a second stem-loop with a mismatched passenger strand (at positions 9-12) for cleavage-independent RISC loading. This bi-functional design is, much more efficient for two reasons; first, the bi-functional promotes guide strand loading onto distinct RISC types, hence promoting mRNA targeting; second, the presence of cleavage-dependent and cleavage-independent RISCs against the same target mRNA promotes silencing by both degradation and translational inhibition/sequestration processes. The potent gene knockdown effector achieves spatial and temporal control by the multiplexed shRNAs under the control of a single pol II promoter. The platform designed by the present inventors mimics the natural process. Multiple studies by others and the literature support the approach of the present inventors. A schematic representation of the bi-functional shRNA design against a single or against multiple targets is shown in FIGS. 7A and 7B, respectively.

Liposomal Delivery System:

The liposomal delivery system involves 1,2-dioleoyl-3-trimethyl-ammoniopropane (DOTAP) and cholesterol. This formulation combines with DNA to form complexes that encapsulate nucleic acids within bilamellar invaginated vesicles (liposomal BIVs). One of the inventors has optimized several features of the BIV delivery system for improved delivery of RNA, DNA, and RNAi plasmids. The liposomal BIVs are fusogenic, thereby bypassing endocytosis mediated DNA cell entry, which can lead to nucleic acid degradation and TLR mediated off-target effects.

The present inventors recognize that an optimized delivery vehicle needs to be a stealthed, which can achieved by PEGylation of nanoparticle with a zeta potential of ≦10 mV for efficient intravascular transport in order to minimize nonspecific binding to negatively-charged serum proteins such as serum albumin (opsonization). Incorporation of targeting moieties such as antibodies and their single chain derivatives (scFv), carbohydrates, or peptides may further enhance transgene localization to the target cell.

The present inventors have created targeted delivery of the complexes in vivo without the use of PEG thereby avoiding an excessively prolonged circulatory half-life. While PEGylation is relevant for DNA or siRNA oligonucleotide delivery to improve membrane permeability, the present inventors recognize that the approach may cause steric hindrance in the BIV liposomal structures, resulting in inefficient DNA encapsulation and reduced gene expression. Furthermore, PEGylated complexes enter the cell predominantly through the endocytic pathway, resulting in degradation of the bulk of the nucleic acid in the lysosomes. While PEG provides extremely long half-life in circulation, this has created problems for patients as exemplified by doxil, a PEGylated liposomal formulation that encapsulates the cytotoxic agent doxorubicin. Attempts to add ligands to doxil for delivery to specific cell surface receptors (e.g. HER2/neu) have not enhanced tumor-specific delivery.

The present invention includes embodiments in which BIVs are produced with DOTAP, and synthetic cholesterol using proprietary manual extrusion process. Furthermore, the delivery was optimized using reversible masking technology. Reversible masking utilizes small molecular weight lipids (about 500 Mol. Wt. and lower; e.g. n-dodecyl-β-D-maltopyranoside) that are uncharged and, thereby, loosely associated with the surface of BIV complexes, thereby temporarily shielding positively charged BIV complexes to bypass non-targeted organs. These small lipids are removed by shear force in the bloodstream. By the time they reach the target cell, charge is re-exposed (optimally ˜45 mV) to facilitate entry.

One reason that the BIV delivery system is uniquely efficient is because the complexes deliver therapeutics into cells by fusion with the cell membrane and avoid the endocytic pathway. The two major entry mechanisms of liposomal entry are via endocytosis or direct fusion with the cell membrane. The inventors found that nucleic acids encapsulated in BIV complexes delivered both in vitro and in vivo enter the cell by direct fusion and that the BIVs largely avoid endosomal uptake, as demonstrated in a comparative study with polyethylene-amine (PEI) in mouse alveolar macrophages. PEI is known to be rapidly and avidly taken up into endosomes, as demonstrated by the localization of >95% of rhodamine labeled oligonucleotides within 2-3 hrs post-transfection.

Cancer targeted delivery with decorated BIVs: The present inventors recognize that siRNAs that are delivered systemically by tumor-targeted nanoparticles (NPs) are significantly more effective in inhibiting the growth of subcutaneous tumors, as compared to undecorated NPs. Targeted delivery does not significantly impact pharmacokinetics or biodistribution, which remains largely an outcome of the EPR (enhanced permeability and retention) effect, but appears to improved transgene expression through enhanced cellular uptake [95-97].

Indeed, a key “missing piece” in development of BIVs for therapeutic is the identification of such non-immunogenic ligands that can be placed on the surface of BIV-complexes to direct them to target cells. While it might be possible to do this with small peptides that are multimerized on the surface of liposomes, these can generate immune responses after repeated injections. Other larger ligands including antibodies, antibody fragments, proteins, partial proteins, etc. are far more refractory than using small peptides for targeted delivery on the surface of liposomes. The complexes of the present invention are thus unique insofar as they not only penetrate tight barriers including tumor vasculature endothelial pores and the interstitial pressure gradient of solid tumors, but also target tumor cells directly. Therefore, the therapeutic approach of the present invention is not limited to delivery solely dependent on the EPR effect but targets the tumor directly.

Small molecules designed to bind proteins selectively can be used with the present invention. Importantly, the small molecules prepared are “bivalent” so they are particularly appropriate for binding cell surface receptors, and resemble secondary structure motifs found at hot-spots in protein-ligand interactions. The present inventors have adapted a strategy to give bivalent molecules that have hydrocarbon tails, and prepared functionalized BIV complexes from these adapted small molecules. An efficient high throughput technology to screen the library was developed and run.

Compacted DNA Nanoparticles:

Safe and Efficient DNA Delivery in Post-Mitotic Cells:

The Copernicus nucleic acid delivery technology is a non-viral synthetic and modular platform in which single molecules of DNA or siRNA are compacted with polycations to yield nanoparticles having the minimum possible volume. The polycations optimized for in vivo delivery is a 10 kDa polyethylene glycol (PEG) modified with a peptide comprising a N-terminus cysteine and 30 lysine residues (CK₃₀PEG10k). The shape of these complexes is dependent in part on the lysine counterion at the time of DNA compaction. The minimum cross-sectional diameter of the rod nanoparticles is 8-11 nm irrespective of the size of the payload plasmid, whereas for ellipsoids the minimum diameter is 20-22 nm for typical expression plasmids. Importantly, these DNA nanoparticles are able to robustly transfect non-dividing cells in culture. Liposome mixtures of compacted DNA generate over 1,000-fold enhanced levels of gene expression compared to liposome naked DNA mixtures. Following in vivo dosing, compacted DNA robustly transfects post-mitotic cells in the lung, brain, and eye. In each of these systems the remarkable ability of compacted DNA to transfect post-mitotic cells appears to be due to the small size of these nanoparticles, which can cross the cross the 25 nm nuclear membrane pore.

One uptake mechanism for these DNA nanoparticles is based on binding to cell surface nucleolin (26 nm K_{D}), with subsequent cytoplasmic trafficking via a non-degradative pathway into the nucleus, where the nanoparticles unravel releasing biologically active DNA. Long-term in vivo expression has been demonstrated for as long as 1 year post-gene transfer. These nanoparticles have a benign toxicity profile and do not stimulate toll-like receptors thereby avoiding toxic cytokine responses, even when the compacted DNA has hundreds of CpG islands and are mixed with liposomes, no toxic effect has been observed [114,115]. DNA nanoparticles have been dosed in humans in a cystic fibrosis trial with encouraging results, with no adverse events attributed to the nanoparticles and with most patients demonstrating biological activity of the CFTR protein [116].

The construction of a novel bi-shRNA therapeutic of the present invention represents a state-of the art approach that can reduce the effective systemic dose needed to achieve an effective therapeutic outcome through post-transcriptional gene knockdown. Effective and clinically applicable delivery approaches are in place that can be rapidly transitioned for systemic targeting of ESFTs.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application Publication No. 2010/0068802 A1: Novel Human Androgen Receptor Alternative Splice Variants as Biomarkers and Therapeutic Targets.
U.S. Patent Application Publication No. 2005/0164970: Identification and Characterization of Cancer Stem Cells and Methods of Use.
U.S. Patent Publication No. 2007/00873529: Inhibitors for Androgen Antagonist Refractory Prostate Cancer.
1. Agoulnik I U, Weigel N L 2006 Androgen receptor action in hormone-dependent and recurrent prostate cancer. J Cell Biochem 99:362-372. PMID: 16619264
2. Agoulnik I U, Vaid A, Bingman W E I, Erdeme H, Frolov A, Smith C L, Ayala G, Ittmann M M, Weigel N L 2005 Role of SRC-1 in the promotion of prostate cancer cell growth and tumor progression. Cancer Res 65:7959-7967. PMID: 16140968
3. Danila D C, Morris M J, De Bono J S, Ryan C J, Denmeade S R, Smith M R, Taplin M E, Bubley G J, Kheoh T, Haqq C, Molina A, Anand A, Koscuiszka M, Larson S M, Schwartz L H, Fleisher M, Scher H I 2010 Phase I I multicenter study of abiraterone acetate plus prednisone therapy in patients with docetaxel-treated castration-resistant prostate cancer. J Clin Oncol 28:1496-1501. PMID: 20159814; PMC3040042
4. Schmidt C 2011 Abiraterone and MVD3100 take androgen deprivation to a new level. J Natl Cancer Inst 103:175-176. PMID: 21242338
5. Scher H I, Beer T M, Higano C S, Anand A, Taplin M E, Efstathiou E, Rathkopf D, Shelkey J, Yu E Y, Alumkal J, Hung D, Hirmand M, Seely L, Morris M J, Danila D C, Humm J, Larson S, Fleisher M, Sawyers C L 2010 Prostate Cancer Foundation/Department of Defense Prostate Cancer Clinical Trials Consortium. Antitumour activity of MDV3100 in castration-resistant prostate cancer: a phase 1-2 study. Lancet 375:1437-1446. PMID: 20398925; PMC2948179
6. Mohler J L, Gregory C W, Ford O H3, Kim D, Weaver C M, Petrusz P, Wilson E M 2004 The androgen axis in recurrent prostate cancer. Clin Cancer Res 10:440-448. PMID: 14760063
7. Culig Z, Hobisch A, Cronauer M V, Radmayr C, Trapman J, Hittmair A, Bartsch G, Klocker H 1994 Androgen receptor activation in prostatic tumor cell lines by insulin-like growth factor-I, keratinocyte growth factor, and epidermal growth factor. Cancer Res 54:5474-5478
8. Desai S J, Ma A H, Tepper C G, Chen H W, Kung H J 2006 Inappropriate activation of the androgen receptor by nonsteroids: involvement of the Src kinase pathway and its therapeutic implications. Cancer Res 66:10449-10459. PMID: 17079466
9. Dehm S M, Schmidt L J, Heemers H V, Vessella R L, Tindall D J 2008 Splicing of a novel androgen receptor exon generates a constitutively active androgen receptor that mediates prostate cancer therapy resistance. Cancer Res 68:5469-5477. PMID: 18593950; PMC2663383
10. Hu R, Dunn T A, Wei S, Isharwal S, Veltri R W, Humphreys E, Han M, Partin A W, Vessella R L, Isaacs W B, Bova G S, Luo J 2009 Ligand-independent androgen receptor variants derived from splicing of cryptic exons signify hormone-refractory prostate cancer. Cancer Res 69:16-22. PMID: 19117982; PMC: 2614301
11. Guo Z, Yang X, Sun F, Jiang R, Linn D E, Chen H, Kong X, Melamed J, Tepper C G, Kung H J, Brodie A M, Edwards J, Qiu Y 2009 A novel androgen receptor splice variant is up-regulated during prostate cancer progression and promotes androgen depletion-resistant growth. Cancer Res 69:2305-2313. PMID: 19244107
12. Sun S, Sprenger C C, Vessella R L, Haugk K, Soriano K, Mostaghei E A, Page S T, Coleman I M, Nguyen H M, Sun H, Nelson P S, Plymate S R 2010 Castration resistance in human prostate cancer is conferred by a frequently occurring androgen receptor splice variant. J Clin Invest 120:2715-2730. PMID: 20644256; PMC2912187
13. Oakes M B, Eyvazzadeh A D, Quint E, Smith Y R 2008 Complete androgen insensitivity syndrome—a review. J Pediatr Adolesc Gynecol 21:305-310. PMID: 19064222
14. Agoulnik I U, Vaid A, Nakka M, Alvarado M, Bingman W E I, Erdem H, Frolov A, Smith C L, Ayala G E, Ittmann M M, Weigel N L 2006 Androgens modulate expression of transcription intermediary factor 2, an androgen receptor coactivator whose expression level correlates with early biochemical recurrence in prostate cancer. Cancer Res 66:10594-10602. PMID: 17079484
15. Agoulnik I U, Weigel N L 2009 Coactivator selective regulation of androgen receptor activity. Steroids 74:669-674. PMID: 19463689; PMC: 2687407
16. Bevan C L, Hoare S, Claessens F, Heery D M, Parker M G 1999 The AF1 and AF2 domains of the androgen receptor interact with distinct regions of SRC1. Mol Cell Biol 19:8383-8392. PMID: 10567563; PMC84931
17. Ma H, Hong H, Huang S-M, Irvine R A, Webb P, Kushner P J, Coetzee G A, Stallcup M R 1999 Multiple signal input and output domains of the 160-kilodalton nuclear receptor coactivator proteins. Mol Cell Biol 19:6164-6173. PMID: 10454563; PMC84548
18. Templeton N S 2010 Liposomes for gene transfer in cancer therapy. Methods Mol Biol 651:271-278. PMID: 20686971
19. Templeton N S, Lasic D D, Frederik P M, Strey H H, Roberts D D, Pavlakis G N 1997 Improved DNA: liposome complexes for increased systemic delivery and gene expression. Nat Biotechnol 15:647-652. PMID: 9219267
20. Shi Q, Nguyen A T, Angell Y, Deng D, Na C R, Burgess K, Roberts D D, Brunicardi F C, Templeton N S 2010 A combinatorial approach for targeted delivery using small molecules and reversible masking to bypass nonspecific uptake in vivo. Gene Ther 17:1085-1097. PMID: 20463761; PMC2923228
21. Templeton N S 2009 Nonviral delivery for genomic therapy of cancer. World J Surg 33:685-697. PMID: 19023615
22. Tirone T A, Fagan S P, Templeton N S, Wang X, Brunicardi F C 2001 Insulinoma-induced hypoglycemic death in mice is prevented with beta cell-specific gene therapy. Ann Surg 233:603-611. PMID: 11323498; PMC1421298
23. Nemunaitis G, Maples P B, Jay C, Gahl W A, Huizing M, Poling J, Tong A W, Phadke A P, Pappen B O, Bedell C, Templeton N S, Kuhn J, Senzer N, Nemunaitis J 2010 Hereditary inclusion body myopathy: single patient response to GNE gene Lipoplex therapy. J Gene Med 12:403-412. PMID: 20440751
24. Lu C 2010 Systemic gene therapy with tumor suppressor FUSI-nanoparticles for recurrent/metastatic lung cancer. J Clin Oncol 28:15s-(suppl; abstract 7582).
25. Rao D D, Maples P B, Senzer N, Kumar P, Wang Z, Pappen B O, Yu Y, Haddock C, Jay C, Phadke A P, Chen S, Kuhn J, Dylewski D, Scott S, Monsma D, Webb C, Tong A, Shanahan D, Nemunaitis J 2010 Enhanced target gene knockdown by a bifunctional shRNA: a novel approach of RNA interference. Cancer Gene Ther 17:780-791. PMID: 20596090
26. Rao D D, Senzer N, Cleary M A, Nemunaitis J 2009 Comparative assessment of siRNA and shRNA off target effects: what is slowing clinical development. Cancer Gene Ther 16:807-809. PMID: 19713999
27. Snoek R, Cheng H, Margiotti K, Wafa L A, Wong C A, Wong E C, Fazli L, Nelson C C, Gleave M E, Rennie P S 2009 In vivo knockdown of the androgen receptor results in growth inhibition and regression of well-established, castration-resistant prostate tumors. Clin Cancer Res 15:39-47. PMID: 19118031
28. Azuma K, Nakashiro K, Sasaki T, Goda H, Onodera J, Tanji N, Yokoyama M, Hamakawa H 2010 Anti-tumor effect of small interfering RNA targeting the androgen receptor in human androgen-independent prostate cancer cells. Biochem Biophys Res Comm 391:1075-1079. PMID: 20004643
29. Quayle S N, Mawji N R, Wang J, Sadar M D 2007 Androgen receptor decoy molecules block the growth of prostate cancer. Proc Natl Acad Sci USA 104:1331-1336. PMID: 17227854; PMC1783142
30. Xu J, Qiu Y, DeMayo F J, Tsai S Y, Tsai M-J, O'Malley B W 1998 Partial hormone resistance in mice with disruption of the steroid receptor coactivator-1 (SRC-1) gene. Science 279:1922-1925. PMID: 9506940
31. Tomlins S A, Rhodes D R, Perner S, Dhanasekaran S M, Mehra R, Sun X W, Varambally S, Cao X, Tchinda J, Kuefer R, Lee C, Montie J E, Shah R B, Pienta K J, Rubin M A, Chinnaiyan A M 2005 Recurrent fusion of TMPRSS2 and ETS transcription factor genes in prostate cancer. Science 310:644-648. PMID: 16254181
32. Blutt S E, McDonnell T J, Polek T C, Weigel N L 2000 Calcitriol-induced apoptosis in LNCaP cells is blocked by overexpression of Bcl-2. Endocrinology 141:10-17. PMID: 10614618
33. Polek T C, Stewart L V, Ryu E J, Cohen M B, Allegretto E A, Weigel N L 2003 p53 is required for 1,25-dihydroxyvitamin D3-induced G0 arrest, but is not required for G1 accumulation or apoptosis of LNCaP prostate cancer cells. Endocrinology 144:50-60. PMID: 12488329
34. Rohan J N, Weigel N L 2009 1 Alpha,25-dihydroxyvitamin D3 reduces c-Myc expression, inhibiting proliferation and causing G1 accumulation in C4-2 prostate cancer cells. Endocrinology 150:2046-2054. PMID: 19164469; PMC2671895
35. Agoulnik I U, Bingman W E I, Nakka M, Li W, Wang Q, Brown M, Liu X S, Weigel N L 2008 Target gene specific regulation of androgen receptor activity by p42/p44 MAPK. Mol Endocrinol 22:2420-2432. PMID: 18787043; PMC2582542.

What is claimed is:

1. A vector comprising:
a first promoter; and
a nucleic acid insert operably linked to the promoter, wherein the insert encodes one or more short hairpin RNAs (shRNA) capable of inhibiting an expression of an Androgen Receptor (AR) gene.

2. The vector of claim 1, wherein the shRNA is a bifunctional shRNA.

3. The vector of claim 1, wherein the shRNA comprises one or more siRNA (cleavage-dependent) and miRNA (cleavage-independent) motifs.

4. The vector of claim 1, wherein the shRNA is both a cleavage-dependent and cleavage-independent inhibitor of the AR gene.

5. The vector of claim 1, wherein a sequence arrangement for the shRNA comprises a 5′ stem arm-19 nucleotide target (AR gene)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′ stem arm.

6. The vector of claim 1, wherein the one or more shRNA correspond to a human and a mouse AR gene, wherein the one or more shRNA are capable of inhibiting an expression of a human and a mouse AR gene.

7. The vector of claim 1, wherein the one or more shRNAs are selected from the group consisting of SEQ ID NO: 10, SEQ ID NO: 11, SEQ ID NO: 12, SEQ ID NO: 13, and any combinations or modifications thereof.

8. The vector of claim 1, further comprising a second nucleic acid insert operably linked to a second promoter, wherein the second insert encodes SRCdp, wherein the SRCdp is capable of blocking the AR-coactivator interface.

9. The vector of claim 8, wherein the first promoter and the second promoter are the same promoter and wherein an optimum gap sequence is intercalated between the first and the second nucleic acid inserts.

10. An expression vector comprising
a promoter; and
a nucleic acid insert operably linked to the promoter, wherein the insert encodes a coactivator-derived peptide (SRCdp), wherein the SRCdp is capable of blocking a AR-coactivator interface.

11. The expression vector of claim 10, wherein SRCdp is derived from SRC-1.

12. The expression vector of claim 10, wherein SRCdp is derived from human or mouse SRC-1 and is capable of blocking a human and a mouse AR-coactivator interface.

13. The expression vector of claim 10, wherein SRCdp comprises amino acids 1050-1240 of SRC-1 comprising SEQ ID NO: 16.

14. The expression vector of claim 10, wherein SRCdp comprises amino acids 1050-1150 of SRC-1 comprising SEQ ID NO: 14.

15. The expression vector of claim 10, wherein SRCdp comprises SEQ ID NO: 15, SEQ ID NO: 17, or both.

16. The expression vector of claim 10, wherein SRCdp is derived from SRC-1, SRC-2, or SRC-3.

17. The expression vector of claim 10, further comprising a nuclear localization signal fused to SRCdp.

18. The expression vector of claim 10, further comprising a second nucleic acid insert operably linked to a promoter, wherein the second insert encodes one or more short hairpin RNAs (shRNA) capable inhibiting an expression of a AR gene.

19. A therapeutic delivery system comprising:
a therapeutic agent carrier; and
a vector that binds to prostate cells comprising
a first nucleic acid insert operably linked to a first promoter or a second nucleic acid insert operably linked to a second promoter or combinations thereof, wherein the first nucleic acid insert encodes one or more short hairpin RNAs (shRNA) capable of inhibiting an expression of a AR gene, wherein the second nucleic acid insert encodes a SRCdp capable of blocking a AR-coactivator interface.

20. The delivery system of claim 19, wherein the first promoter and the second promoter is the same promoter and wherein an optimum gap sequence is intercalated between the first and the second nucleic acid inserts.

21. The delivery system of claim 19, wherein the therapeutic agent carrier is a nanoparticle capable of compacting DNA.

22. The delivery system of claim 21, wherein the nanoparticles comprise one or more polycations.

23. The delivery system of claim 21, wherein the compacted DNA nanoparticles are further encapsulated in a liposome.

24. The delivery system of claim 23, wherein the liposome is a bilamellar invaginated vesicle (BIV).

25. The delivery system of claim 23, wherein the liposome is a reversibly masked liposome.

26. The delivery system of claim 23, wherein the liposome is decorated with one or more “smart” receptor targeting moieties.

27. The delivery system of claim 26, wherein the one or more “smart” receptor targeting moieties are small molecule bivalent beta-turn mimics.

28. The delivery system of claim 19, wherein the delivery system is adapted for use to suppress tumor cell growth, treat prostate cancer, or both in a human or animal subject.

29. The delivery system of claim 19, wherein the delivery system is used to suppress tumor cell growth, treat prostate cancer, or both by itself or in combination with one or more chemotherapeutic agents, radiation therapy, surgical intervention, antibody therapy, Vitamin D, or any combinations thereof.

30. The delivery system of claim 19, further comprising one or more 10 kDA polyethylene glycol (PEG)-substituted cysteine-lysine 3-mer (CK30PEG10k) peptides.

31. A method to deliver a vector to a tissue comprising:
providing a vector comprising a first nucleic acid insert operably linked to a first promoter, or a second nucleic acid insert operably linked to a second promoter, or combinations thereof, wherein the first nucleic acid insert encodes one or more short hairpin RNAs (shRNA) capable of inhibiting an expression of an AR gene and wherein the second nucleic acid insert encodes a SRCdp capable of blocking a AR-coactivator interface;
combining the expression vector with a therapeutic agent carrier; and
administering a therapeutically effective amount of the expression vector and therapeutic agent carrier complex to a patient in need thereof.

32. The delivery system of claim 31, wherein the first promoter and the second promoter is the same promoter and wherein an optimum gap sequence is intercalated between the first and the second nucleic acid inserts.

33. The method of claim 31, wherein the therapeutic agent carrier is a nanoparticle capable of compacting DNA.

34. The method of claim 33, wherein the DNA nanoparticle is compacted with one or more polycations, wherein the one or more polycations comprise a 10 kDA polyethylene glycol (PEG)-substituted cysteine-lysine 3-mer peptide (CK30PEG10k) or a 30-mer lysine condensing peptide.

35. The method of claim 33, wherein the compacted DNA nanoparticles are further encapsulated in a liposome, wherein the liposome is a bilamellar invaginated vesicle (BIV)

36. The method of claim 35, wherein the liposome is a reversible masked liposome.

37. The method of claim 35, where the liposome is decorated with one or more “smart” receptor targeting moieties.

38. The method of claim 35, wherein the one or more “smart” receptor targeting moieties are small molecule bivalent beta-turn mimics.

39. The vector of claim 31, wherein the one or more shRNAs are selected from the group consisting of SEQ ID NO: 10, SEQ ID NO: 11, SEQ ID NO: 12, SEQ ID NO: 13, and any combinations or modifications thereof.

40. The expression vector of claim 31, wherein the SRCdp insert comprises SEQ ID NO: 15, SEQ ID NO: 17, or both.

41. A method of suppressing a tumor cell growth, treating prostate cancer, or both in a human subject comprising the steps of:
identifying the human subject in need for suppression of the tumor cell growth, treatment of prostate cancer or both; and
administering a vector in a therapeutic agent carrier complex to the human subject in an amount sufficient to suppress the tumor cell growth, treat prostate cancer or both, wherein the vector comprises a first nucleic acid insert operably linked to a first promoter, or a second nucleic acid insert operably linked to a second promoter, or combinations thereof, wherein the first nucleic acid insert encodes one or more short hairpin RNAs (shRNA) capable of inhibiting an expression of a AR gene and wherein the second nucleic acid insert encodes a SRCdp, wherein the SRCdp is capable of blocking a AR-coactivator interface, wherein inhibition of AR expression or blockage of the AR-coactivator interface reduces tumor growth.

42. The method of claim 41, further comprising the step of administering the vector before, after, or concurrently as a combination therapy with one or more treatment methods selected from the group consisting of chemotherapy, radiation therapy, surgical intervention, antibody therapy, Vitamin D therapy, or any combinations thereof.

43. The method of claim 41, wherein the therapeutic agent carrier is a nanoparticle capable of compacting DNA or a reversibly masked liposome decorated with one or more “smart” receptor targeting moieties.

44. The method of claim 43, wherein the DNA nanoparticle is compacted with one or more polycations, wherein the one or more polycations is a 10 kDA polyethylene glycol (PEG)-substituted cysteine-lysine 3-mer peptide (CK30PEG10k) or a 30-mer lysine condensing peptide.

45. The method of claim 43, wherein the reversibly masked liposome is a bilamellar invaginated vesicle (BIV).

46. The method of claim 43, wherein the one or more “smart” receptor targeting moieties are small molecule bivalent beta-turn mimics.

47. The method of claim 43, wherein the compacted DNA nanoparticles are further encapsulated in a liposome.

48. The method of claim 41, wherein the shRNA is a bifunctional shRNA.

49. The method of claim 48, wherein the bifunctional shRNA incorporates siRNA (cleavage-dependent) and miRNA (cleavage-independent) motifs.

50. The method of claim 48, wherein the bifunctional shRNA is both a cleavage-dependent and a cleavage-independent inhibitor of AR gene expression.

51. The method of claim 41, wherein a sequence arrangement for the shRNA comprises a 5′ stem arm-19 nucleotide target (AR gene)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′ stem arm.

52. The method of claim 41, wherein the shRNA is selected from the group consisting of SEQ ID NO: 10, SEQ ID NO: 11, SEQ ID NO: 12, SEQ ID NO: 13, and any combinations or modifications thereof.

53. The method of claim 41, wherein SRCdp is derived from SRC-1.

54. The method of claim 41, wherein SRCdp is derived from human or mouse SRC-1 and is capable of blocking a human and a mouse AR-coactivator interface.

55. The method of claim 41, wherein SRCdp comprises amino acids 1050-1240 of SRC-1 comprising SEQ ID NO: 16.

56. The method of claim 41, wherein SRCdp comprises amino acids 1050-1150 of SRC-1 comprising SEQ ID NO: 14.

57. The method of claim 41, wherein SRCdp is selected from the group consisting of SEQ ID NO: 15, SEQ ID NO: 17, or both.

58. The method of claim 41, wherein SRCdp is derived from SRC-1, SRC-2, or SRC-3.

59. The method of claim 41, further comprising a nuclear localization signal fused to SRCdp.

60. The method of claim 41, wherein the tumor cell growth is an androgen dependent prostate cancer.

61. The method of claim 41, wherein the tumor cell growth is an androgen independent prostate cancer.

62. A method for studying biological and clinical manifestations of a current or proposed anti-cancer therapeutic strategy in a human or animal subject, customizing anti-cancer therapy for an individual human or animal subject, or both in a human or animal subject comprising the step of:
identifying the human or animal subject in need of screening for reactions to an anti-cancer medication, customization of the anti-cancer therapy, or both;
administering a vector in a therapeutic agent carrier complex to the human or animal subject in an amount sufficient to suppress the tumor cell growth, cancer or both, wherein the vector comprises a first nucleic acid insert operably linked to a first promoter, or a second nucleic acid insert operably linked to a second promoter, or combinations thereof, wherein the first nucleic acid insert encodes one or more short hairpin RNAs (shRNA) capable of inhibiting an expression of a AR gene and wherein the second nucleic acid insert encodes a SRCdp, wherein the SRCdp is capable of blocking a AR-coactivator interface, wherein inhibition of AR expression or blockage of the AR-coactivator interface reduces tumor growth;
collecting biological and clinical information from the human or animal subject after administration of the vector in the therapeutic agent complex; and
making a decision to terminate, continue, or modify a current or proposed anti-cancer therapeutic strategy in the human or animal subject based on the biological or clinical information, wherein the therapeutic strategy comprises administration of the vector in the therapeutic agent carrier by itself or in combination chemotherapy, radiation therapy, surgical intervention, antibody therapy, Vitamin D therapy, or any combinations thereof.

63. The method of claim 62, wherein the cancer is prostate cancer.


SEQUENCE LISTING (22 sequences)

SEQ ID NO: 1 - DNA, 4314 bp, artificial sequence
  synthetic oligonucleotide
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaac

SEQ ID NO: 2 - DNA, 19 bp, artificial sequence
  synthetic oligonucleotide
cagaaatgattgcactatt

SEQ ID NO: 3 - DNA, 19 bp, Artificial sequence
  synthetic oligonucleotide
aatagtgcaatcatttctg

SEQ ID NO: 4 - DNA, 19 bp, Artificial sequence
  Synthetic oligonucleotide
cagcctgttgaactcttct

SEQ ID NO: 5 - DNA, 19 bp, Artificial sequence
  Synthetic oligonucleotide
agaagagttcaacaggctg

SEQ ID NO: 6 - DNA, 19 bp, Artificial sequence
  Synthetic oligonucleotide
acgaggcagctgcgtacca

SEQ ID NO: 7 - DNA, 19 bp, artificial sequence
  shynthetic oligonucleotide
tggtacgcagctgcctcgt

SEQ ID NO: 8 - DNA, 19 bp, Artificial sequence
  Synthetic oligonucleotide
gcaggaagcagtatccgaa

SEQ ID NO: 9 - DNA, 19 bp, artificial sequence
  Synthetic oligonucleotide
ttcggatactgcttcctgc

SEQ ID NO: 10 - DNA, 245 bp, artificial sequence
  synthetic oligonucleotide
tcgactgctgttgaagtgagcgcccagaaatgattgcactatttagtgaagccacagatgtaaatagtgcaatcatttctggttgcctactgcctcggaagcagctcactacattactcagctgttgaagtgagcgcccagaaatgtcagcaccatttagtgaagccacagatgtaaatagtgcaatcatttctggttgcctactgcctcggaagcttaataaaggatcttttattttcattggc

SEQ ID NO: 11 - DNA, 245 bp, artificial sequence
  Synthetic oligonucleotide
tcgactgctgttgaagtgagcgcccagcctgttgaactcttcttagtgaagccacagatgtaagaagagttcaacaggctggttgcctactgcctcggaagcagctcactacattactcagctgttgaagtgagcgcccagcctgtatcactattcttagtgaagccacagatgtaagaagagttcaacaggctggttgcctactgcctcggaagcttaataaaggatcttttattttcattggc

SEQ ID NO: 12 - DNA, 245 bp, artificial sequence
  Synthetic oligonucleotide
tcgactgctgttgaagtgagcgccacgaggcagctgcgtaccatagtgaagccacagatgtatggtacgcagctgcctcgtgttgcctactgcctcggaagcagctcactacattactcagctgttgaagtgagcgccacgaggcactagcgtatcatagtgaagccacagatgtatggtacgcagctgcctcgtgttgcctactgcctcggaagcttaataaaggatcttttattttcattggc

SEQ ID NO: 13 - DNA, 245 bp, artificial sequence
  Synthetic oligonucleotide
tcgactgctgttgaagtgagcgccgcaggaagcagtatccgaatagtgaagccacagatgtattcggatactgcttcctgcgttgcctactgcctcggaagcagctcactacattactcagctgttgaagtgagcgccgcaggaagttctatcagaatagtgaagccacagatgtattcggatactgcttcctgcgttgcctactgcctcggaagcttaataaaggatcttttattttcattggc

SEQ ID NO: 14 - protein, 102 aa, artificial sequence
  Synthetic peptide
MAPNQLRLQLQQRLQGQQQLIHQNRQAILNQFAATAPVGINMRSGMQQQITPQPPLNAQMLAQRQRELYSQQHRQRQLIQQQRAMLMRQQSFGNNLPPSSGL

SEQ ID NO: 15 - DNA, 309 bp, artificial sequence
  Synthetic oligonucleotide
atggcacctaaccagcttcgacttcaactacagcagcgattacagggacaacagcagttgatacaccaaaatcggcaagctatcttaaaccagtttgcagcaactgctcctgttggcatcaatatgagatcaggcatgcaacagcaaattacacctcagccacccctgaatgctcaaatgttggcacaacgtcagcgggaactgtacagtcaacagcaccgacagaggcagctaatacagcagcaaagagccatgcttatgaggcagcaaagctttgggaacaacctccctccctcatctggactatag

SEQ ID NO: 16 - protein, 192 aa, artificial sequence
  Synthetic peptide
MAPNQLRLQLQQRLQGQQQLIHQNRQAILNQFAATAPVGINMRSGMQQQITPQPPLNAQMLAQRQRELYSQQHRQRQLIQQQRAMLMRQQSFGNNLPPSSGLPVQMGNPRLPQGAPQQFPYPPNYGTNPGTPPASTSPFSQLAANPEASLANRNSMVSRGMTGNIGGQFGTGINPQMQQNVFQYPGAGMVPQ

SEQ ID NO: 17 - DNA, 579 bp, artificial sequence
  Synthetic oligonucleotide
atggcacctaaccagcttcgacttcaactacagcagcgattacagggacaacagcagttgatacaccaaaatcggcaagctatcttaaaccagtttgcagcaactgctcctgttggcatcaatatgagatcaggcatgcaacagcaaattacacctcagccacccctgaatgctcaaatgttggcacaacgtcagcgggaactgtacagtcaacagcaccgacagaggcagctaatacagcagcaaagagccatgcttatgaggcagcaaagctttgggaacaacctccctccctcatctggactaccagttcaaatggggaacccccgtcttcctcagggtgctccacagcaattcccctatccaccaaactatggtacaaatccaggaaccccacctgcttctaccagcccgttttcacaactagcagcaaatcctgaagcatccttggccaaccgcaacagcatggtgagcagaggcatgacaggaaacataggaggacagtttggcactggaatcaatcctcagatgcagcagaatgtcttccagtatccaggagcaggaatggttccccaatag

SEQ ID NO: 18 - protein, 10 aa, artificial sequence
  Synthetic peptide
DDLPRRRGRS

SEQ ID NO: 19 - protein, 112 aa, artificial sequence
  Synthetic oligonucleotide
MDDLPRRRGRSAPNQLRLQLQQRLQGQQQLIHQNRQAILNQFAATAPVGINMRSGMQQQITPQPPLNAQMLAQRQRELYSQQHRQRQLIQQQRAMLMRQQSFGNNLPPSSGL

SEQ ID NO: 20 - DNA, 339 bp, artificial sequence
  Synthetic oligonucleotide
atggacgacctgcccagaagaagaggcagatccgcacctaaccagcttcgacttcaactacagcagcgattacagggacaacagcagttgatacaccaaaatcggcaagctatcttaaaccagtttgcagcaactgctcctgttggcatcaatatgagatcaggcatgcaacagcaaattacacctcagccacccctgaatgctcaaatgttggcacaacgtcagcgggaactgtacagtcaacagcaccgacagaggcagctaatacagcagcaaagagccatgcttatgaggcagcaaagctttgggaacaacctccctccctcatctggactatag

SEQ ID NO: 21 - protein, 202 aa, artificial sequence
  synthetic peptide
MDDLPRRRGRSAPNQLRLQLQQRLQGQQQLIHQNRQAILNQFAATAPVGINMRSGMQQQITPQPPLNAQMLAQRQRELYSQQHRQRQLIQQQRAMLMRQQSFGNNLPPSSGLPVQMGNPRLPQGAPQQFPYPPNYGTNPGTPPASTSPFSQLAANPEASLANRNSMVSRGMTGNIGGQFGTGINPQMQQNVFQYPGAGMVPQ

SEQ ID NO: 22 - DNA, 609 bp, artificial sequence
  Synthetic oligonucleotide
atggacgacctgcccagaagaagaggcagatccgcacctaaccagcttcgacttcaactacagcagcgattacagggacaacagcagttgatacaccaaaatcggcaagctatcttaaaccagtttgcagcaactgctcctgttggcatcaatatgagatcaggcatgcaacagcaaattacacctcagccacccctgaatgctcaaatgttggcacaacgtcagcgggaactgtacagtcaacagcaccgacagaggcagctaatacagcagcaaagagccatgcttatgaggcagcaaagctttgggaacaacctccctccctcatctggactaccagttcaaatggggaacccccgtcttcctcagggtgctccacagcaattcccctatccaccaaactatggtacaaatccaggaaccccacctgcttctaccagcccgttttcacaactagcagcaaatcctgaagcatccttggccaaccgcaacagcatggtgagcagaggcatgacaggaaacataggaggacagtttggcactggaatcaatcctcagatgcagcagaatgtcttccagtatccaggagcaggaatggttccccaatag